(19) United States
(12) Patent Application Publication
(10) Pub. No.: US 2013/0274352 A1
(43) Pub. Date: Oct. 17, 2013

(54) Oral Drug Devices and Drug Formulations

(71) Applicant: The Regents of the University of California, Oakland, CA (US)

(72) Inventors: Kathryn Whitehead, Pittsburgh, PA (US); Natalie Karr, San Francisco, CA (US); Anubhav Arora, Carlsbad, CA (US); Samir Mitragotri, Santa Barbara, CA (US); Vivek Gupta, Goleta, CA (US)

(21) Appl. No.: 13/802,079

(22) Filed: Mar. 13, 2013

Related U.S. Application Data
(63) Continuation-in-part of application No. 13/264,585, filed on Oct. 14, 2011.
(60) Provisional application No. 61/169,171, filed on Apr. 14, 2009.

(51) Int. Cl. A61K 9/00

(52) U.S. Cl. CPC A61K 9/006
USPC 514/788; 514/772

(57) ABSTRACT

Compositions containing a drug to be delivered and at least one chemical permeation enhancer (CPE), and methods of making and using these compositions are described herein. In a preferred embodiment, the compositions contain two or more CPEs which behave in synergy to increase the permeability of the epithelium, while providing an acceptably low level of cytotoxicity to the cells. The concentration of the one or more CPEs is selected to provide the greatest amount of overall potential (OP). Additionally, the CPEs are selected based on the treatment. CPEs that behave primarily by transcellular transport are preferred for delivering drugs into epithelial cells. CPEs that behave primarily by paracellular transport are preferred for delivering drugs through epithelial cells. Also provided herein are mucoadhesive oral dosage forms. In a preferred embodiment, the oral dosage form is a multi-compartmental device, containing (i) a supporting compartment, (ii) drug compartment and (iii) mucoadhesive compartment.

### CROSS-REFERENCE TO RELATED APPLICATION

This application is a continuation in part of U.S. Ser. No. 13/264,585, filed Oct. 14, 2011, which is a §371 application of PCT/US2010/031047, filed Apr. 14, 2010, which is a non-provisional application of U.S. Ser. No. 61/169,171, filed Apr. 14, 2009, the disclosures of which are herein incorporated by reference in their entirety.

### GOVERNMENT SUPPORT

This invention was made with government support under a fellowship to Kathryn Whitehead from the Graduate Research and Education in Adaptive bio-Technology (GREAT) Training Program by the University of California Biotechnology Research and Education Program. The government has certain rights in the invention.

### FIELD OF THE INVENTION

The field of the invention is drug delivery formulations and devices and methods for making and using these formulations and devices.

### BACKGROUND OF THE INVENTION

Oral delivery is a highly sought-after means of drug administration due to its convenience and positive effect on patient compliance. However, the oral route cannot be utilized for the delivery of proteins and other macromolecules due to enzymatic degradation in the gastrointestinal tract and limited transport across the intestinal epithelium. (see e.g., M. Goldberg and I. Gomez-Orellana, Nat Rev Drug Discov. 2:289-295 (2003); and G. Mustata and S. M. Dinh, Crit Rev Ther Drug Carrier Syst. 23:111-135 (2006)). While the former issue is being tackled by innovative encapsulation strategies and enzyme inhibitors, the latter can potentially be addressed by using chemicals to promote drug uptake across the epithelium (see B. J. Aungst, J Pharm Sci. 89:429-442 (2000)).

Chemical permeation enhancers (CPEs) aid oral drug absorption by altering the structure of the cellular membrane (transcellular route) and/or the tight junctions between cells (paracellular route) of the intestinal epithelium (Salama, et al., Adv Drug Deliv Rev. 58:15-28 (2006); and Bourdet, et al., Pharm Res., 23:1178-1187 (2006)). Unfortunately, many reports indicate that enhancer efficacy is often linked to toxicity (E. S. Swenson, et al., Pharm Res. 11:1132-1142 (1994); and R. Konsoula & F. A. Barile, Toxicol In Vitro, 19:675-684 (2005)). It is commonly believed that oral permeation enhancers are either ‘potent and toxic’ or ‘weak and safe’. As a result, permeation enhancers are not widely used in oral formulations.

The full potential of CPEs for oral delivery remains unclear since there is no fundamental understanding of the principles that govern enhancer behavior. Specifically, it is unclear whether the experimentally observed correlation between the potency and toxicity of CPEs is intrinsic in nature or whether it is a consequence of the limited conditions of previous studies. Additionally, little awareness exists as to how chemical category and concentration can influence the interplay between potency and toxicity. Further, the mechanism by which individual enhancers and combinations of CPEs increase drug permeability is unclear.

Chemical permeation enhancers aid drug uptake through two distinct mechanisms, both of which involve the mediation of a physical cellular barrier. The passive transcellular route involves the alteration of the structure of the cell membrane, whereas an enhancement of the paracellular route entails an opening of the tight junctions between epithelial cells (Salama, et al., Adv Drug Deliv Rev. 58:15-28 (2006); and Bourdet, et al., Pharm Res. 23:1178-1187 (2006)). Numerous methods have been used to make mechanistic assessments, including fluorescence microscopy (see Chao, et al., J Pharm Sci, 87:1395-1399 (1998)), immunostaining (see T. Suzuki & H. Hara, Life Sciences, 79:401-410 (2006); and E. Duizer, et al., J Pharmacol Exp Ther, 287:395-402 (1998)), voltage clamping (Hess, et al., Eur J Pharm Sci, 25:307-312 (2005); and Uchiyama, et al., J Pharm Pharmacol, 51:1241-1250 (1999)), and permeability studies (Maher, et al., Pharm Res, 24:1336-1345 (2007); and Sharma, et al., Il Farmaco, 60:870-873 (2005)). Unfortunately, these techniques are often used inconsistently across laboratories, and mechanistic analysis tends to be incomplete. Specifically, enhancer mechanism is typically considered to be solely transcellular or paracellular, and the ability of an enhancer to affect both routes remains largely unexplored.

Due to the narrow scope of the existing data on CPE potency and toxicity and the irreconcilable differences in experimental models and test conditions, these critical questions previously have gone unanswered.

In addition to delivery to the intestinal mucosa, drug delivery to other mucosal surfaces is in need of improved formulations.

Some oral dosage forms present particular challenges for the delivery of poorly absorbed molecules, enzyme-sensitive bioactive agents or drugs that require site-specific targeting delivery. For these bioactive agents or drugs, particular strategies are needed to achieve sufficient drug absorption into the blood stream. In prior conventional methods, particles such as liposomes, micro/nanoparticles or micro/nanocapsules are often used as drug carriers to overcome the poor bioavailabilities of these drugs. Additionally, by coating mucoadhesive polymers onto the surface of the particles, these particles can easily adhere to intestine mucus and therefore prolong their migration time and extend release of the drug.

However, there are some limitations to the existing particle systems. Specifically, i) drug release is not unidirectional, therefore a portion of the released drug is lost into the luminal fluid and is not delivered directly to the site; ii) transit of particles in the gastrointestinal (GI) tract is often highly variable; and iii) as the particle surface is exposed to intestinal fluid, bioactive agents encapsulated in these particles are generally not sufficiently protected to prevent proteolytic degradation.

Therefore it is an object of the invention to provide improved formulations for drug delivery through or within mucosal surfaces.

It is a further object of the invention to provide improved oral drug delivery devices.

It is a further object of the invention to provide a method for selecting chemical permeation enhancers for drug delivery formulations through or within mucosal surfaces.

It is a further object of the invention to provide means to stimulate the gastrointestinal tract by application of energy.

It is a further object of the invention to remove undesired molecules from the body, and particularly from the gastrointestinal tract.

### SUMMARY OF THE INVENTION

Compositions containing a drug to be delivered and at least one chemical permeation enhancer (CPE), and methods of making and using these compositions are described herein. In a preferred embodiment, the compositions contain two or more CPEs which behave in synergy to increase the permeability of the epithelium, while providing an acceptably low level of cytotoxicity to the cells. The concentration of the one or more CPEs may be selected to provide the greatest amount of overall potential (OP). Additionally, the one or more CPEs are selected based on the disease or disorder to be treated. CPEs which behave primarily by transcellular transport are preferred for delivering drugs into epithelial cells. In contrast, CPEs which behave primarily by paracellular transport are preferred for delivering drugs through epithelial cells.

Also provided herein are oral dosage forms. In a preferred embodiment, the oral dosage form is a multi-compartmental device, preferably containing three compartments: (i) a supporting compartment (110), (ii) drug compartment (120) and (iii) mucoadhesive compartment (130). The device adheres to the intestine (140) and delivers drugs directly to the wall of the intestine.

### BRIEF DESCRIPTION OF THE DRAWINGS

FIG. 1 is a graph of mean enhancement potential (EP) versus mean toxicity potential (TP) data for all of the 153 enhancer formulations (51 enhancers at 3 concentrations each) tested (n=3-6 for the formulations tested). Error bars are not provided in the figure for clarity. Mean standard deviations are 0.07 and 0.09 for EP and TP values, respectively.

FIGS. 2A-C are graphs of EP (circles) and TP (squares) versus concentration (% w/v) for three (3) enhancer formulations: sodium deoxycholate (FIG. 2A), the sodium salt of oleic acid (FIG. 2B), and sodium laureth sulfate (FIG. 2C). FIG. 2D is a graph of overall potential (OP) versus concentration (% w/v) for sodium deoxycholate (squares with dashed line), the sodium salt of oleic acid (diamonds with dashed line), and sodium laureth sulfate (circles with solid line).

FIG. 3 is a graph of EP vs. LDH potential (LP) values for all of the 153 enhancer formulations (51 enhancers at 3 concentrations each) tested (n=3-6). Error bars are not provided in the figure for clarity. Mean standard deviations are 0.07 and 0.12 for EP and LP values, respectively.

FIG. 4 is a bar graph of average K values for each of the eleven (11) chemical categories (averaged for all enhancers and concentrations within each category). Category abbreviations are: anionic surfactants (AS), cationic surfactants (CS), zwitterionic surfactants (ZS), nonionic surfactants (NS), bile salts (BS), fatty acids (FA), fatty esters (FE), fatty amines (FM), sodium salts of fatty acids (SS), nitrogen-containing rings (NR), and others (OT). Error bars indicate standard deviation (i.e. the extent to which enhancers within the same category affect the same route).

FIG. 5 is a graphical representation of synergy in a binary system, containing decyltrimethyl ammonium bromide (DTAB) and sodium laureth sulfate (SLA). TP values are shown for combinations of SLA and DTAB at a total concentration of 0.1% as a function of weight fraction SLA (n=6). The dotted line represents ‘expected’ values of TP based on a linear average of individual components.

FIG. 6A is a graph of the all of the TP values for the 1210 binary enhancer combinations tested. FIG. 6B is a bar graph of the distribution of synergy values (S) for the 1210 binary enhancer combinations tested.

FIG. 7A is a graph of EP versus TP for the top 25 binary enhancer combinations tested (closed circles). Error bars reflect the standard deviation (n=3-6). FIG. 7B is a bar graph of the distribution of OP values for the top 25 binary formulations, with OP=1 corresponding to an ideal permeation enhancer (maximum efficacy, minimal cytotoxicity).

FIG. 8A is a graph of the all of the TP values for the 264 ternary enhancer combinations tested. FIG. 8B is a bar graph of the distribution of synergy values (S) for the 264 ternary enhancer combinations tested.

FIG. 9 is an illustration of a hemispherical multicompartmental device for mucosal delivery.

FIG. 10 is an illustration of a hemispherical multicompartmental device for mucosal delivery with the opposite orientation as the orientation of the device in FIG. 9.

FIG. 11 is an illustration of a multicompartmental device, where the drug is distributed in several compartments (320a, b, c, and d).

FIGS. 12A and B are illustrations of a flexible device multicompartmental device (410) that is sufficiently flexible to be rolled inside a capsule (420).

FIG. 13 is an illustration of a device comprising an electrode, which is activated by a battery.

FIGS. 14A and B are illustrations of a flanged multicompartmental device. This device contains a hemispherical multicompartmental portion, which is connected to a flange (150) of the mucoadhesive compartment (130).

FIG. 15 is an illustration of a microsphere-containing hemispherally shaped device. Microspheres loaded with drugs are used as drug compartments (160a, b, and c). These microspheres are encapsulated in a supporting compartment (110) wherein the supporting compartment holds the microspheres together. The microspheres rest on a mucoadhesive compartment (130) that supports the adhesion of the device on mucosa.

FIGS. 16A, B and C are illustrations of a device that has flanges (710a, b, c, and d) that fold onto themselves to prevent adhesion of devices to each other.

FIG. 17 is a graph of % BZK in formulation versus LC50/minimum inhibitory concentration (MIC) for six formulations containing BZK and S20 (n=3) MIC was measured by incubating the formulations in B. thailendensis and LC50 was measured by incubating the formulations in epidermal keratinocyte cultures. The figure shows that mixtures of BZK and S20 had higher LC50/MIC ratio compared to either BZK or S20.

FIG. 18 is a graph of transepithelial electrical resistance (TEER) values (% of initial value) over time (hours) following incubation with varying concentrations of palmityldimethyl ammonio propane sulfonate (PPS) over time (hours). Circles: No PPS, Triangles: 0.01% w/v PPS, Diamonds: 0.03% w/v.

FIGS. 19A and B are graphs of TEER values (FIG. 19A) and Enhancement Ration (FIG. 19B) of FITC-insulin transport in the present of PPS across Caco-2 monolayers. FIG. 19A is a graph of TEER values (% of initial value) over time (hours) following incubation with varying concentrations of PPS. FITC-insulin was loaded in apical chambers with 2 different PPS concentrations of 0.01% w/v (triangles), and 0.03% w/v (diamond). Open circles denote FITC-insulin control. FIG. 19B is a bar graph comparing the enhancement ratio of cumulative transport of FITC-insulin across Caco-2 monolayers in presence of PPS (no PPS, 0.01% w/v PPS and 0.03% w/v PPS). Data represent mean±SD (n=3) of three individual experiments.

FIG. 20 is a bar graph TEER reversibility after exposure to 0.03% w/v PPS comparing two different time periods. Caco-2 monolayers were exposed to 0.03% w/v PPS in the apical chamber for 10 minutes (striped bars) and 1 hr (black bars).

FIG. 21 is a graph of % cell viability versus concentration of PPS (% w/v)×100 for Caco-2 cell monolayers exposed to various concentrations of PPS for three different time periods. Caco-2 cells were grown in a 96-well plate, and exposed to concentrations of PPS (0.0005%-0.03% w/v) for various times including 10 min (triangles), 1 hr (circles), and 5 hrs (squares). Data represent mean±SD (n=8).

FIG. 22 is a bar graph of the calculated fluorescence intensity of FITC-insulin permeation enhancement by PPS based on images taken by confocal laser scanning microscopy experiments. All scale bars represent 40 μm. Data represent mean±SD (n=3).

FIGS. 23A and 23B are graphs of the in vivo efficacy of PPS in enhancing intestinal absorption of Salmon Calcitonin (sCT). sCT solution (with or without PPS) was administered in the duodenal region. FIG. 23A is a graph of the pharmacodynamic efficacy of sCT (with or without PPS) in reducing plasma calcium concentration. Data are plotted as % reduction in plasma calcium over time (hours). FIG. 23B is a graph of the plasma concentration of sCT following duodenal administration (with or without PPS) depicting plasma concentration of sCT (ng/ml) over time (hours). Data represent mean±SD (n=3-4) of three individual experiments. For both FIGS. 23A and 23B, sCT alone (3 mg/kg; open circles), sCT solution with 0.1% PPS (closed circles), and sCT solution with 1% PPS (open squares).

### DETAILED DESCRIPTION OF THE INVENTION

### I. Definitions

As used herein “chemical permeation enhancer” or “CPE” generally means a chemical that aids transport across the epithelium by altering the structure of the cellular membrane (transcellular route) and/or the tight junctions between cells (paracellular route) of the epithelium.

As used herein, “drug” refers to chemical or biological molecules providing a therapeutic, diagnostic, or prophylactic effect in vivo.

As used herein “enhancement potential” or “EP” refers to the permeability increase due to exposure to one or more CPEs as compared to the permeability increase due to exposure to a positive control through a Caco-2 monolayer after 10 minutes of exposure to the CPE(s) or positive control, as measured by transepithelial electrical resistance (TEER) measurements (Millicell-ERS voltohmmeter, Millipore, Billerica, Mass.). The Examples described herein used 1% Triton X-100 as the positive control.

All TEER values were normalized by their initial values. EP was calculated as the reduction in TEER of a Caco-2 monolayer after 10 minutes of exposure to that CPE, normalized to the reduction in TEER after exposure to the positive control, 1% Triton X-100:

$$EP = \frac{100 \% - {TEER}_{CPE}}{100 \% - {TEER}_{+}} \qquad Eq . 1$$

where TEER_{CPE} and TEER₊ are the resistance values (% of initial) of the enhancer solution and positive control solution, respectively, after 10 minutes of exposure. EP lies on a scale of 0 to 1, with 1 representing maximum enhancement as compared to the positive control.

As used herein “toxicity potential” or “TP” is used to assess the safety of CPEs and refers to the toxicity of one or more CPEs as determined using a Methyl Thiazole Tetrazolium (MTT) kit (American Type Culture Collection, Rockville, Md.). Caco-2 cells were seeded at 10⁵ cells/well onto a 96-well plate. Enhancer solutions (100 μl) were applied for 30 minutes. 10 μl of reagent from an MTT kit (American Type Culture Collection, Rockville, Md.) was applied to each well for 5 hours, after which 100 μl of detergent was applied to each well and allowed to incubate in the dark at room temperature for about 40 hours. Absorbance was read at 570 nm (MTT dye) and 650 nm (detergent).

TP values are reported as the fraction of nonviable cells, as compared to the negative control, DMEM. TP values range from 0 to 1, with 0 indicating no mitrochondrial toxicity, and 1 representing maximum toxicity.

As used herein “overall potential” or “OP” refers to the difference between EP and TP:

OP=EP−TP, where −1<OP<1 Eq. 2

Although higher OP values typically indicate increased potential for use, EP and TP values should also be considered in conjunction with OP values when assessing a CPE or combination of CPEs.

As used herein “synergy” or “S” refers to the difference between the linear average of the toxicity of the individual components and the experimentally measured toxicity of the mixture. Synergy was calculated as follows:

S=[X₁·TP₁+X₂·TP₂+X₃·TP₃]−TPₘᵢₓ Eq. 3

where X₁, X₂, and X₃ are the weight fractions of single enhancers 1, 2, and 3, respectively, and TP₁, TP₂, TP₃, and TPₘᵢₓ are the toxicity potentials of pure CPE 1, pure CPE 2, pure CPE 3, and the mixture of CPEs at the corresponding weight fractions X₁, X₂, and X₃. All TP values in the equation above are obtained at the same total concentration. Since TP values can range from 0 to 1, maximum and minimum Synergy values are 1 and −1, respectively.

### II. Drug-Containing Compositions for Targeted Drug Delivery

The compositions contain one or more CPE(s) and a drug to be delivered. The compositions may be used to administer a wide range of drugs to a variety of mucosal surfaces.

A. Chemical Permeation Enhancers

The CPE or combination of CPEs are selected to have high potency, relatively low toxicity and aid drug uptake via a transcellular or paracellular route, or both, depending on the disease or disorder to be treated.

CPEs possess a broad range of chemical structures. Many CPEs are small molecules. Chemical categories of such CPEs include: anionic surfactants (AS), cationic surfactants (CS), zwitterionic surfactants (ZS), nonionic surfactants (NS), bile salts (BS), fatty acids (FA), fatty esters (FE), fatty amines (FM), sodium salts of fatty acids (SS), nitrogen-containing rings (NR), and others (OT). A list of exemplary CPEs within each of these categories is provided in Table 1.

TABLE 1
List of Exemplary Chemical Permeation Enhancers

| | | | CAS |
|---|---|---|---|
| Abbreviation | Chemical Name | Category | Number |
| SLS | Sodium lauryl sulfate | AS | 151-21-3 |
| SDS | Sodium decyl sulfate | AS | 142-87-0 |
| SOS | Sodium octyl sulfate | AS | 142-31-4 |
| SLA | Sodium laureth sulfate | AS | 68585-34-2 |
| NLS | N-Lauryl sarcosinate | AS | 137-16-6 |
| CTAB | Cetyltrimethyl ammonium | CS | 57-09-0 |
| | bromide | | |
| DTAB | Decyltrimethyl ammonium | CS | 2082-84-0 |
| | bromide | | |
| BDAC | Benzyldimethyl dodecyl | CS | 139-07-1 |
| | ammonium chloride | | |
| TTAC | Myristyltrimethyl ammonium | CS | 4574-04-3 |
| | chloride | | |
| DPC | Dodecyl pyridinium chloride | CS | 104-74-5 |
| DPS | Decyldimethyl ammonio | ZS | 15163-36-7 |
| | propane sulfonate | | |
| MPS | Myristyldimethyl ammonio | ZS | 14933-09-6 |
| | propane sulfonate | | |
| PPS | Palmityldimethyl ammonio | ZS | 2281-11-0 |
| | propane sulfonate | | |
| CBC | ChemBetaine CAS | ZS | N/A |
| | | | (mixture) |
| CBO | ChemBetaine Oleyl | ZS | N/A |
| | | | (mixture) |
| PCC | Palmitoyl carnitine chloride | ZS | 6865-14-1 |
| IP | Nonylphenoxypolyoxyethylene | NS | 68412-54-4 |
| T20 | Polyoxyethylene sorbitan | NS | 9005-64-5 |
| | monolaurate | | |
| T40 | Polyoxyethylene sorbitan | NS | 9005-66-7 |
| | monopalmitate | | |
| SP80 | Sorbitan monooleate | NS | 1338-43-8 |
| TX100 | Triton-X 100 | NS | 9002-93-1 |
| SDC | Sodium deoxycholate | BS | 302-95-4 |
| SGC | Sodium glycocholate | BS | 863-57-0 |
| CA | Cholic acid | FA | 73163-53-8 |
| HA | Hexanoic acid | FA | 142-91-6 |
| HPA | Heptanoic acid | FA | 111-14-8 |
| LME | Methyl laurate | FE | 111-82-0 |
| MIE | Isopropyl myristate | FE | 110-27-0 |
| IPP | Isopropyl palmitate | FE | 142-91-6 |
| MPT | Methyl palmitate | FE | 112-39-0 |
| SDE | Diethyl sebaccate | FE | 110-40-7 |
| SOA | Sodium oleate | SS | 143-19-1 |
| UR | Urea | FM | 57-13-6 |
| LAM | Lauryl amine | FM | 124-22-1 |
| CL | Caprolactam | NR | 105-60-2 |
| MP | Methyl pyrrolidone | NR | 872-50-4 |
| OP | Octyl pyrrolidone | NR | 2687-94-7 |
| MPZ | Methyl piperazine | NR | 109-01-3 |
| PPZ | Phenyl piperazine | NR | 92-54-6 |
| EDTA | Ethylenediaminetetraacetic | OT | 10378-23-1 |
| | acid | | |
| SS | Sodium salicylate | OT | 54-21-7 |
| CP | Carbopol 934P | OT | 9003-04-7 |
| GA | Glyccyrhetinic acid | OT | 471-53-4 |
| BL | Bromelain | OT | 9001-00-7 |
| PO | Pinene oxide | OT | 1686-14-2 |
| LM | Limonene | OT | 5989-27-5 |
| CN | Cineole | OT | 470-82-6 |
| ODD | Octyl dodecanol | OT | 5333-42-6 |
| FCH | Fenchone | OT | 7787-20-4 |
| MTH | Menthone | OT | 14073-97-3 |
| TPMB | Trimethoxy propylene methyl | OT | 2883-98-9 |
| | benzene | | |
| | polysorbate 20 (TWEEN 20) | NS | 9005-64-5 |

1. Preferred Categories of CPEs

In the preferred embodiment, the CPE has a high EP (i.e. greater than 0.5) and low TP (i.e. less than 0.5). Preferably the CPE has an OP of greater than 0, more preferably the CPE has an OP of greater than 0.5, most preferably the CPE has an OP of approximately 1.

Compounds containing nitrogen-containing rings, zwitterionic surfactants, cationic surfactants, fatty amines, and anionic surfactants are preferred categories for CPEs. In a preferred embodiment, the compounds containing nitrogen-containing rings are members of the piperazine family, such as phenyl piperazine (PPZ). In another preferred embodiment, the CPE is a zwitterionic surfactant, such as palmityldimethyl ammonio propane sulfonate (PPS).

2. Concentrations

As depicted in the Examples provided herein, the concentration of the one or more CPEs in the drug-containing composition typically has a strong effect on the ability of the CPEs to increase permeability of the drug across a given mucosal surface.

The concentration of the one or more CPEs in a drug containing composition is effective to increase the rate of absorption of the drug at a site of delivery, relative to rate of absorption of the drug at the same site in the absence of the chemical permeation enhancer, without causing one or more symptoms associated with malfunctions of the gastrointestinal tract, such as gastrointestinal discomfort, interference in digestion, or other symptoms, such as bloating, abdominal pain, cramping, constipation, bleeding, and/or diarrhea. The concentration of the CPE is effective to increase the rate of drug absorption, without causing necrosis or specific inflammation at the site of delivery.

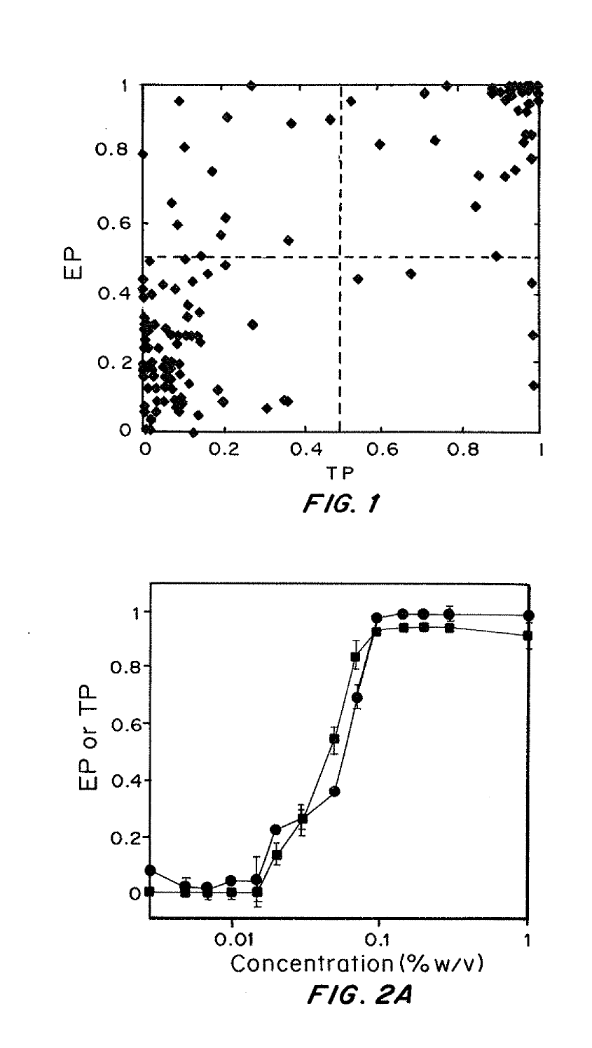

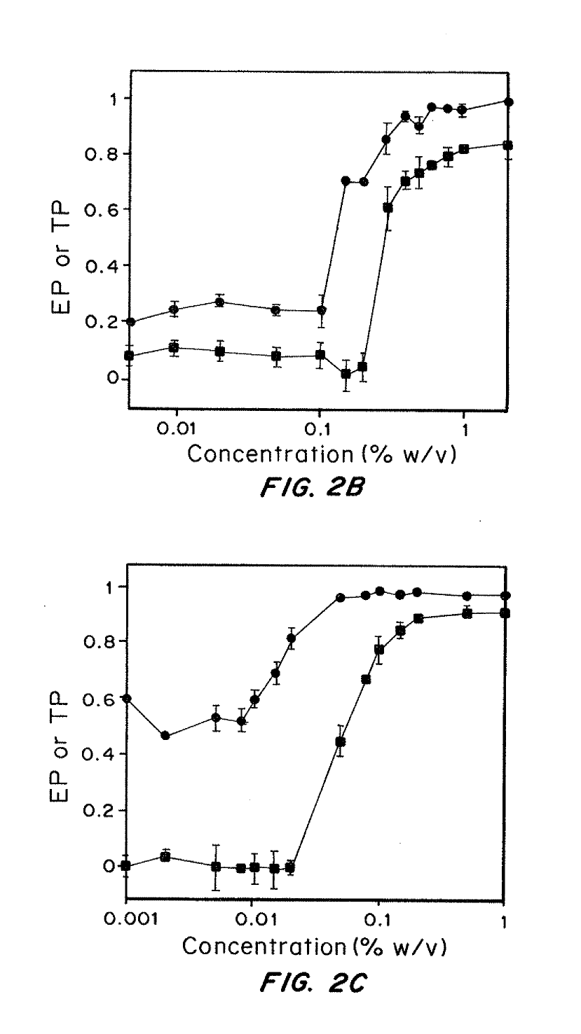

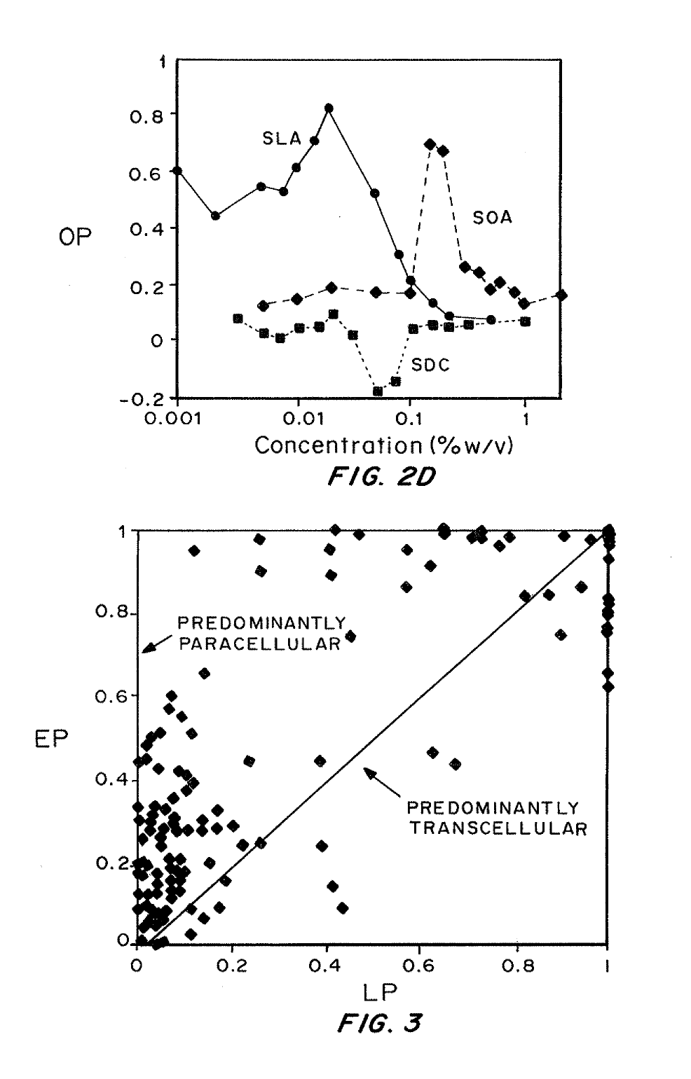

The concentration of the CPE may be determined through a combination of in vitro and in vivo tests. An enhancer's potential therapeutic concentration window corresponds with the concentrations at which the enhancer's EP is sufficiently greater than the enhancer's TP to (1) result in an OP greater than zero and (2) produce the highest values of OP, which correspond with a peak in a graph of concentration (% w/v) versus OP. An exemplary graph is provided in FIG. 2D. The width of the peak in OP corresponds to the range of an enhancer's potential therapeutic concentration window. Preferably, the concentration of CPE in the formulation ranges from about 0.01% (w/v) to about 10% (w/v). In some embodiments, the concentration of CPE in the formulation ranges from about 0.01% (w/v) to about 5% (w/v), or from about 0.01% to about 2% (w/v), or from about 0.01% to about 1% (w/v). The particular therapeutic concentration window for each CPE can be determined and used to select the concentration or concentration range that is therapeutically effective in vivo.

Determining the concentration range that is therapeutically effective in vivo in humans (and other mammals) involves routine testing. The potential therapeutic concentration window determined using in vitro tests described in Example 1, provides a starting point for this determination. It is expected that the therapeutic concentrations required in vivo will be greater than the potential therapeutic concentration window determined via in vitro tests. This increase in concentration for the CPE is likely needed to account for the presence of serum and mucus proteins in vivo, which interact with the CPE and dilute the effective concentration of the CPE when it is administered to a patient. This is demonstrated in Example 5, which tests a higher concentration of the CPE PPS in rats than was determined via the in vitro experiment described in Example 1 and determined that even at this higher concentration the CPE did not cause necrosis or specific inflammation at the site of delivery.

3. Synergistic Combinations of CPEs

In a preferred embodiment, the drug-containing composition includes two or more CPEs, where the CPEs are synergistic enhancer formulations. The term “synergistic enhancer formulations” or “SEFs” as used herein refers to those combinations of CPEs with a Synergy (S) value that is greater than 0.25 (S>0.25).

As noted in Equation 3 and as demonstrated in Example 3, the value of S is a function of the weight percent of each CPE in the formulation.

Table 2 lists ten safe and potent combinations of CPEs along with their corresponding S values.

TABLE 2
10 Safe and Potent SEFs

| CPE 1 | CPE 2 | CPE 3 | X₁ | X₂ | X₃ | Conc. (%) | OP | S |
|---|---|---|---|---|---|---|---|---|
| SLA | DTAB | CBC | 5 | 2 | 3 | 0.1 | 0.99 | 0.58 |
| SLA | DTAB | CBC | 5 | 3 | 2 | 0.1 | 0.96 | 0.53 |
| HAM | CBC | — | 1 | 9 | — | 0.1 | 0.95 | 0.60 |
| HAM | SLA | CBC | 2 | 3 | 5 | 0.1 | 0.95 | 0.58 |
| HAM | SLA | — | 7 | 3 | — | 0.1 | 0.94 | 0.44 |
| HAM | CBC | — | 4 | 6 | — | 0.1 | 0.94 | 0.51 |
| HAM | SLA | CBC | 3 | 3 | 4 | 0.1 | 0.94 | 0.61 |
| HAM | SLA | DTAB | 1 | 6 | 3 | 0.1 | 0.93 | 0.67 |
| SLA | DTAB | CBC | 7 | 2 | 1 | 0.1 | 0.93 | 0.57 |
| SLA | DTAB | CBC | 7 | 1 | 2 | 0.1 | 0.91 | 0.56 |

Preferred SEFs typically contain one or more of the following enhancers: sodium laureth sulfate (SLA), decyltrimethyl ammonium bromide (DTAB), chembetaine (CBC), or hexylamine (HAM). The most preferred SEFs are listed above in Table 2.

CPEs may be polymers, including polycations such as polyethyleneimine, polylysine and polyarginine, polyanions such as polyacrylic acid or any other polymer that can sufficiently permeabilize the epithelium including carbopol, pectin and other mucoadhesive polymers. The CPE may also be a peptide, such as cell-permeating peptides that are capable of penetrating the epithelial membranes, polyarginine or other peptides that specifically bind to the epithelium and increase its permeability. The CPE may also be a protein that is known to enhance the permeability of the epithelium by disrupting the membrane, opening the tight junctions and/or facilitating transcytosis.

B. Drugs

The drug-containing compositions may contain any suitable drug. The drug is selected based on the disease or disorder to be treated or prevented. The drug can be a small molecule or macromolecule, such as a protein or peptide. In the preferred embodiment the drug is a protein or peptide. However, a wide range of drugs may be included in the compositions. Drugs contemplated for use in the formulations described herein include, but are not limited to, the following categories and examples of drugs and alternative forms of these drugs such as alternative salt forms, free acid forms, free base forms, and hydrates:

analgesics/antipyretics (e.g., aspirin, acetaminophen, ibuprofen, naproxen sodium, buprenorphine, propoxyphene hydrochloride, propoxyphene napsylate, meperidine hydrochloride, hydromorphone hydrochloride, morphine, oxycodone, codeine, dihydrocodeine bitartrate, pentazocine, hydrocodone bitartrate, levorphanol, diflunisal, trolamine salicylate, nalbuphine hydrochloride, mefenamic acid, butorphanol, choline salicylate, butalbital, phenyltoloxamine citrate, diphenhydramine citrate, methotrimeprazine, cinnamedrine hydrochloride, and meprobamate);
antiasthamatics (e.g., ketotifen and traxanox);
antibiotics (e.g., neomycin, streptomycin, chloramphenicol, cephalosporin, ampicillin, penicillin, tetracycline, and ciprofloxacin);
antidepressants (e.g., nefopam, oxypertine, doxepin, amoxapine, trazodone, amitriptyline, maprotiline, phenelzine, desipramine, nortriptyline, tranylcypromine, fluoxetine, doxepin, imipramine, imipramine pamoate, isocarboxazid, trimipramine, and protriptyline);
antidiabetics (e.g., biguanides and sulfonylurea derivatives);
antifungal agents (e.g., griseofulvin, ketoconazole, itraconizole, amphotericin B, nystatin, and candicidin);
antihypertensive agents (e.g., propanolol, propafenone, oxyprenolol, nifedipine, reserpine, trimethaphan, phenoxybenzamine, pargyline hydrochloride, deserpidine, diazoxide, guanethidine monosulfate, minoxidil, rescinnamine, sodium nitroprusside, rauwolfia serpentina, alseroxylon, and phentolamine);
anti-inflammatories (e.g., (non-steroidal) indomethacin, ketoprofen, flurbiprofen, naproxen, ibuprofen, ramifenazone, piroxicam, (steroidal) cortisone, dexamethasone, fluazacort, celecoxib, rofecoxib, hydrocortisone, prednisolone, and prednisone);
antineoplastics (e.g., cyclophosphamide, actinomycin, bleomycin, daunorubicin, doxorubicin, epirubicin, mitomycin, methotrexate, fluorouracil, carboplatin, carmustine (BCNU), methyl-CCNU, cisplatin, etoposide, camptothecin and derivatives thereof, phenesterine, paclitaxel and derivatives thereof, docetaxel and derivatives thereof, vinblastine, vincristine, tamoxifen, and piposulfan);
antianxiety agents (e.g., lorazepam, buspirone, prazepam, chlordiazepoxide, oxazepam, clorazepate dipotassium, diazepam, hydroxyzine pamoate, hydroxyzine hydrochloride, alprazolam, droperidol, halazepam, chlormezanone, and dantrolene);
immunosuppressive agents (e.g., cyclosporine, azathioprine, mizoribine, and FK506 (tacrolimus));
antimigraine agents (e.g., ergotamine, propanolol, isometheptene mucate, and dichloralphenazone);
sedatives/hypnotics (e.g., barbiturates such as pentobarbital, pentobarbital, and secobarbital; and benzodiazapines such as flurazepam hydrochloride, triazolam, and midazolam);
antianginal agents (e.g., beta-adrenergic blockers; calcium channel blockers such as nifedipine, and diltiazem; and nitrates such as nitroglycerin, isosorbide dinitrate, pentaerythritol tetranitrate, and erythrityl tetranitrate);
antipsychotic agents (e.g., haloperidol, loxapine succinate, loxapine hydrochloride, thioridazine, thioridazine hydrochloride, thiothixene, fluphenazine, fluphenazine decanoate, fluphenazine enanthate, trifluoperazine, chlorpromazine, perphenazine, lithium citrate, and prochlorperazine);
antimanic agents (e.g., lithium carbonate);
antiarrhythmics (e.g., bretylium tosylate, esmolol, verapamil, amiodarone, encamide, digoxin, digitoxin, mexiletine, disopyramide phosphate, procainamide, quinidine sulfate, quinidine gluconate, quinidine polygalacturonate, flecamide acetate, tocamide, and lidocaine);
antiarthritic agents (e.g., phenylbutazone, sulindac, penicillamine, salsalate, piroxicam, azathioprine, indomethacin, meclofenamate, gold sodium thiomalate, ketoprofen, auranofin, aurothioglucose, and tolmetin sodium);
antigout agents (e.g., colchicine, and allopurinol);
anticoagulants (e.g., heparin, heparin sodium, and warfarin sodium);
thrombolytic agents (e.g., urokinase, streptokinase, and alteplase);
antifibrinolytic agents (e.g., aminocaproic acid);
hemorheologic agents (e.g., pentoxifylline);
antiplatelet agents (e.g., aspirin);
anticonvulsants (e.g., valproic acid, divalproex sodium, phenyloin, phenyloin sodium, clonazepam, primidone, phenobarbitol, carbamazepine, amobarbital sodium, methsuximide, metharbital, mephobarbital, mephenyloin, phensuximide, paramethadione, ethotoin, phenacemide, secobarbitol sodium, clorazepate dipotassium, and trimethadione);
antiparkinson agents (e.g., ethosuximide);
antihistamines/antipruritics (e.g., hydroxyzine, diphenhydramine, chlorpheniramine, brompheniramine maleate, cyproheptadine hydrochloride, terfenadine, clemastine fumarate, triprolidine, carbinoxamine, diphenylpyraline, phenindamine, azatadine, tripelennamine, dexchlorpheniramine maleate, methdilazine, and);
agents useful for calcium regulation (e.g., calcitonin, and parathyroid hormone);
antibacterial agents (e.g., amikacin sulfate, aztreonam, chloramphenicol, chloramphenicol palmitate, ciprofloxacin, clindamycin, clindamycin palmitate, clindamycin phosphate, metronidazole, metronidazole hydrochloride, gentamicin sulfate, lincomycin hydrochloride, tobramycin sulfate, vancomycin hydrochloride, polymyxin B sulfate, colistimethate sodium, and colistin sulfate);
antiviral agents (e.g., interferon alpha, beta or gamma, zidovudine, amantadine hydrochloride, ribavirin, and acyclovir);
antimicrobials (e.g., cephalosporins such as cefazolin sodium, cephradine, cefaclor, cephapirin sodium, ceftizoxime sodium, cefoperazone sodium, cefotetan disodium, cefuroxime e azotil, cefotaxime sodium, cefadroxil monohydrate, cephalexin, cephalothin sodium, cephalexin hydrochloride monohydrate, cefamandole nafate, cefoxitin sodium, cefonicid sodium, ceforanide, ceftriaxone sodium, ceftazidime, cefadroxil, cephradine, and cefuroxime sodium; penicillins such as ampicillin, amoxicillin, penicillin G benzathine, cyclacillin, ampicillin sodium, penicillin G potassium, penicillin V potassium, piperacillin sodium, oxacillin sodium, bacampicillin hydrochloride, cloxacillin sodium, ticarcillin disodium, azlocillin sodium, carbenicillin indanyl sodium, penicillin G procaine, methicillin sodium, and nafcillin sodium; erythromycins such as erythromycin ethylsuccinate, erythromycin, erythromycin estolate, erythromycin lactobionate, erythromycin stearate, and erythromycin ethylsuccinate; and tetracyclines such as tetracycline hydrochloride, doxycycline hyclate, and minocycline hydrochloride, azithromycin, clarithromycin);
anti-infectives (e.g., GM-CSF);
bronchodilators (e.g., sympathomimetics such as epinephrine hydrochloride, metaproterenol sulfate, terbutaline sulfate, isoetharine, isoetharine mesylate, isoetharine hydrochloride, albuterol sulfate, albuterol, bitolterolmesylate, isoproterenol hydrochloride, terbutaline sulfate, epinephrine bitartrate, metaproterenol sulfate, epinephrine, and epinephrine bitartrate; anticholinergic agents such as ipratropium bromide; xanthines such as aminophylline, dyphylline, metaproterenol sulfate, and aminophylline; mast cell stabilizers such as cromolyn sodium; inhalant corticosteroids such as beclomethasone dipropionate (BDP), and beclomethasone dipropionate monohydrate; salbutamol; ipratropium bromide; budesonide; ketotifen; salmeterol; xinafoate; terbutaline sulfate; triamcinolone; theophylline; nedocromil sodium; metaproterenol sulfate; albuterol; flunisolide; fluticasone proprionate;
steroidal compounds, hormones and hormone analogues (e.g., incretins and incretin mimetics such as GLP-1 and exenatide, androgens such as danazol, testosterone cypionate, fluoxymesterone, ethyltestosterone, testosterone enathate, methyltestosterone, fluoxymesterone, and testosterone cypionate; estrogens such as estradiol, estropipate, and conjugated estrogens; progestins such as methoxyprogesterone acetate, and norethindrone acetate; corticosteroids such as triamcinolone, betamethasone, betamethasone sodium phosphate, dexamethasone, dexamethasone sodium phosphate, dexamethasone acetate, prednisone, methylprednisolone acetate suspension, triamcinolone acetonide, methylprednisolone, prednisolone sodium phosphate, methylprednisolone sodium succinate, hydrocortisone sodium succinate, triamcinolone hexacetonide, hydrocortisone, hydrocortisone cypionate, prednisolone, fludrocortisone acetate, paramethasone acetate, prednisolone tebutate, prednisolone acetate, prednisolone sodium phosphate, and hydrocortisone sodium succinate; and thyroid hormones such as levothyroxine sodium);
hypoglycemic agents (e.g., human insulin, purified beef insulin, purified pork insulin, recombinantly produced insulin, insulin analogs, glyburide, chlorpropamide, glipizide, tolbutamide, and tolazamide);
hypolipidemic agents (e.g., clofibrate, dextrothyroxine sodium, probucol, pravastitin, atorvastatin, lovastatin, and niacin);
peptides;
proteins (e.g., DNase, alginase, superoxide dismutase, and lipase);
nucleic acids (e.g., sense or anti-sense nucleic acids encoding any therapeutically useful protein, including any of the proteins described herein, and siRNA);
agents useful for erythropoiesis stimulation (e.g., erythropoietin);
antiulcer/antireflux agents (e.g., famotidine, cimetidine, and ranitidine hydrochloride);
antinauseants/antiemetics (e.g., meclizine hydrochloride, nabilone, prochlorperazine, dimenhydrinate, promethazine hydrochloride, thiethylperazine, and scopolamine);
oil-soluble vitamins (e.g., vitamins A, D, E, K, and the like);
as well as other drugs such as mitotane, halonitrosoureas, anthrocyclines, and ellipticine.

A description of these and other classes of useful drugs and a listing of species within each class can be found in Martindale, The Extra Pharmacopoeia, 30th Ed. (The Pharmaceutical Press, London 1993), the disclosure of which is incorporated herein by reference in its entirety.

In one embodiment, the drug is a CPE. For example, many CPEs possess antimicrobial properties. Examples of such CPEs include cationic surfactants and cationic polymers. However, their use for microbicidal applications is limited by their cytotoxicity. This issue can be mitigated by combining such CPEs with other non-toxic CPEs. For example, a combination of a cationic surfactant, benzalkoniium chloride (BZK) and sorbitan monolaurate (S20) provides an optimum balance between the potency and toxicity. Other combinations where mixing CPEs to mitigate toxicity without significantly compromising potency may also be used.

In one embodiment, the drug may be an enzyme or a neutralizing agent. In this embodiment, the drug is not intended to be delivered across the epithelium, rather it remains within the device and draws undesired molecules from the blood across the epithelium into the device and neutralizes the undesired molecule for the purpose of detoxification. Examples of undesired molecules to be removed from the body include alcohol, urea, neurotoxins or any other molecule that has undesired effect on the body.

B. Excipients

Drug-containing compositions may be prepared using a pharmaceutically acceptable carrier composed of materials that are considered safe and effective and may be administered to an individual without causing undesirable biological side effects or unwanted interactions. The carrier is all components present in the pharmaceutical formulation other than the active drug and the CPE(s).

Suitable excipients are determined based on a number of factors, including the dosage form, desired release rate of the drug, stability of the drug to be delivered.

Excipients include, but are not limited to, polyethylene glycols, humectants, vegetable oils, medium chain mono, di and triglycerides, lecithin, waxes, hydrogenated vegetable oils, colloidal silicon dioxide, polyvinylpyrrolidone (PVP) (“povidone”), celluloses, CARBOPOL® polymers (Lubrizol Advanced Materials, Inc.) (i.e. crosslinked acrylic acid-based polymers), acrylate polymers, other hydrogel forming polymers, plasticizers, crystallization inhibitors, bulk filling agents, solubilizers, bioavailability enhancers and combinations thereof.

C. Dosage Forms

Any dosage form suitable for delivery to the desired mucosal surface, including mucosa of the intestine, nasal cavity, oral cavity, colon, rectum, and vagina, may be used. For oral dosage forms for delivery to the intestinal mucosa, the drug-containing compositions may be in the form of tablets, mini-tab, multiparticulates (including micro- and nano-particles), osmotic delivery systems capsules, patches, and liquids.

For delivery to the buccal mucosa, suitable dosage forms include, but are not limited to films, tablets, and patches.

For delivery to the nasal mucosa, suitable dosage forms include, but are not limited to, dried powders, creams, gels, and aerosols.

For delivery to the rectal mucosa, suitable dosage forms include, but are not limited to, dried powders, creams, gels, and aerosols.

For delivery to the vaginal mucosa, suitable dosage forms include, but are not limited to, dried powders, suppositories, ovuals, creams, gels, and aerosols.

In one embodiment, one or more chemical permeation enhancers are delivered to a mucosal surface by a drug delivery device containing a reservoir for holding the chemical permeation enhancer(s). In a preferred embodiment, the reservoir also contains one or more drug(s). The majority, but not all, of the surface of the reservoir is coated with a protective coating. In the portion of the surface of the reservoir without the protective coating, the surface is covered with a bioadhesive layer for adhering the device to a mucosal surface. At least one side of the device is substantially permeable, and at least another side of the device is substantially impermeable; this directs the delivery of the chemical permeation enhancer(s) and, optionally, drug(s). In a preferred embodiment, the dimensions of the device include at least one dimension between 100 micrometer and 5 millimeter and two dimensions between 100 micrometer and 2 millimeter.

In another embodiment, the CPEs are contained within a drug delivery device. A variety of different devices having a variety of different geometries and structures may be formed. Preferably the device is a multicompartment device, such as described below in Section III, which also contains one or more CPEs.

In another embodiment, the oral dosage form contains a matrix, which includes at least one drug and one or more chemical permeation enhancer(s) dispersed therein. A majority, but not all, of the surface of the matrix is coated with a protective coating. Optionally a portion of the surface of the matrix is coated with a bioadhesive layer. In a preferred embodiment the portions of the matrix that are coated with the protective coating are substantially impermeable, and the portions that are not coated with the protective coating are substantially permeable. This allows for unidirectional release of the drug(s) and chemical permeation enhancer(s).

Devices for oral drug delivery may be formed using bioadhesive, biocompatible and biodegradable materials. In one embodiment, the devices are mixture of a Carbopol polymer, pectin and a modified cellulose, such as Carbopol 934 (BF Goodrich Co., Cleveland, Ohio), pectin (Sigma Chemicals, St. Louis, Mo.), and sodium carboxylmethylcellulose (SCMC, Aldrich, Milwaukee, Wis.). The weight percent of each material in the mixture can be varied to achieve different mucoadhesive effects. In one embodiment, the weight ratio of Carbopol:pectin:SCMC is 1:1:2. The drug to be delivered is added to the mixture in an appropriate amount to achieve the desired dosage. Then the mixture is compressed using a hydraulic press. The pressure used during this step can be varied to affect the dissolution time of the device in vivo. Then a hole punch can be used to cut this disk into smaller disks, such as disks with radii of 1-4 mm. In order to protect the devices from proteolytic degradation in the intestinal lumen, these disks are coated with ethylcellulose on all but one side. For example a solution of 5% w/v ethylcellulose (Sigma Chemicals, St. Louis, Mo.) in acetone may be used. This procedure produces an impermeable ethylcellulose layer on all but one side of the device, and ensures the unidirectional release of the drug from the device.

Optionally, the drug-containing device can be encapsulated in a capsule, such as a gelatin capsule.

### II. Multicompartment Devices for Oral Drug Delivery

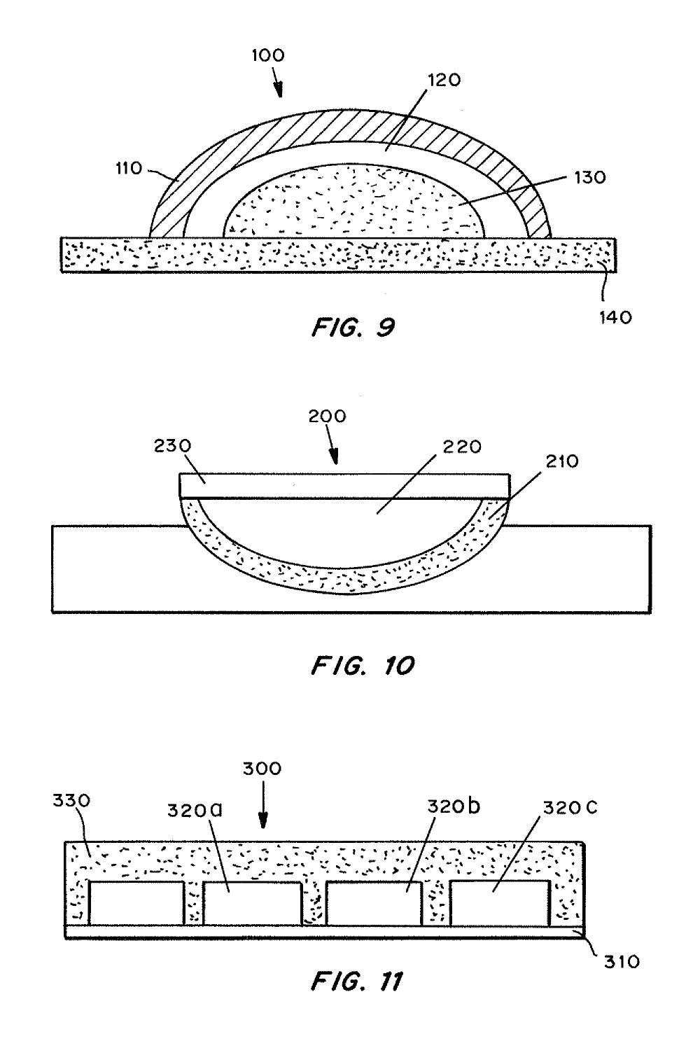

In one embodiment, the device is hemispherical in shape (see e.g., FIGS. 9 and 10). As shown in FIG. 9, the device (100) may be a multicompartmental device that contains a mucoadhesive compartment (130) that exhibits strong adhesion on a mucosal membrane (140). The mucoadhseive compartment is backed by a drug compartment (120) comprising a drug along with one or more suitable excipients. The drug compartment is backed by the supporting layer (110). The hemispherical shape of the device is selected to reduce undesired interactions between the devices which can lead to aggregation prior to adhesion of the devices on the mucosal surface.

In another embodiment, the order of the layers in the device (200) is reversed so that the mucosadhesive compartment (210) is hemispherically shaped, while the supporting layer (230) is substantially flat, with the drug compartment (220) located between the mucoadhesive compartment and the supporting layer (230) (see FIG. 10).

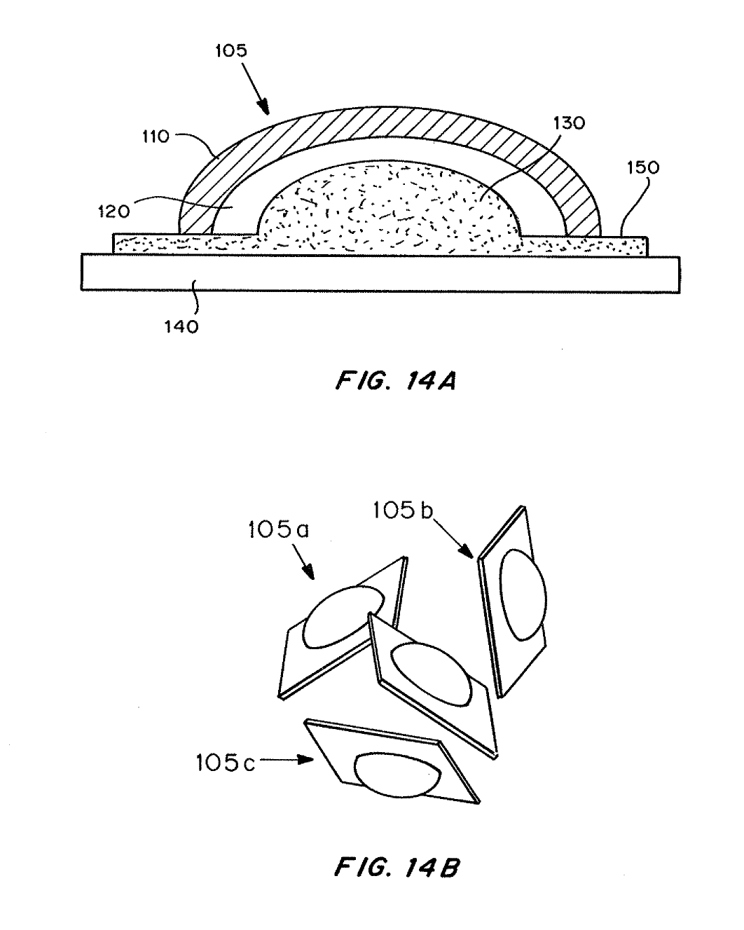

Optionally, the device contains a multicompartmental hemispherical portion (100), as illustrated in FIG. 9, which is attached to a mucoadhesive compartment (130) that extends past the diameter of the hemisphere and forms a flange (150) (see FIGS. 14A and B). The flange forming mucoadhesive compartment is particularly useful in improving the adhesion of the device on a mucosal surface.

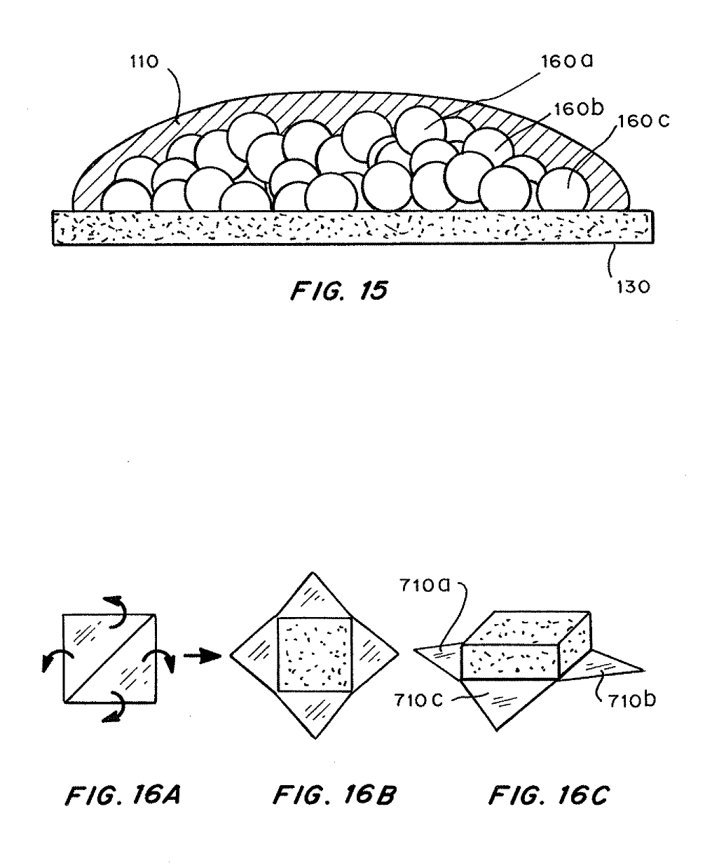

In another embodiment, the hemispherical device depicted by FIG. 9 can be modified so that the device contains multiple microspheres, which contain one or more drugs, in place of a single drug compartment. As shown in FIG. 15, the microspheres are loaded with drugs and serve as multiple drug compartments (160a, b and c). The microspheres are encapsulated in a supporting compartment (110) that retains the microspheres within the device. The microspheres rest on a mucoadhesive compartment (130), which adheres to mucosa. The microspheres (160a, b, c) may remain within the supporting compartment (110) for the duration of delivery. Alternatively, the microspheres may be released from the device where they migrate through the gastrointestinal tract and perform drug delivery. The function of the microspheres may be enhanced by engineering their structure. In one embodiment, the microspheres may possess a disk-like or a rod-like shape, which facilitates their adhesion on the mucosal surface due to enhanced surface contact area. In another embodiment, the microsphere may possess multiple distinct internal regions to facilitate its adhesion and protection of the drug and the one or more CPEs.

In another embodiment, the device is a multicompartment device (300) where the drug is distributed in several compartments (320a, b, and c) (see FIG. 11). Compartmentalization of the drug results in more even distribution of the drug compared to the same device with a single drug compartment. In one embodiment, each compartment contains the same drug. Optionally, each compartment contains the same dosage. Alternatively, each compartment may contain different concentrations of the same drug, preferably one compartment contains a higher drug concentration than a compartment that is adjacent to it. This embodiment may be useful in improving update of the drug following its release from the device.

In another embodiment, one or more of the compartments contain a different drug from the drug in the remaining compartment(s).

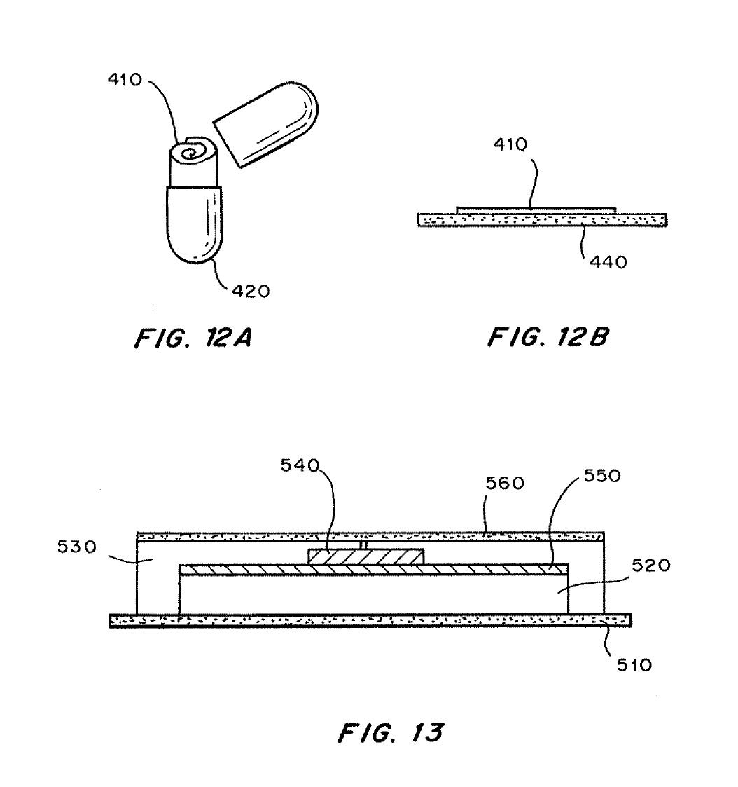

In another embodiment, the multicompartmental device is sufficiently flexible to be rolled and placed within a capsule for oral drug delivery. An example of this device is illustrated in FIGS. 12A and B. Rolling makes it possible to put an otherwise large device (410) (as illustrated in FIG. 12B) into a manageable size capsule (420) for oral drug delivery. After the patient swallows the capsule and as the capsule travels through the gastrointestinal tract, the capsule will degrade allowing for the release of the multicompartmental device. Upon exiting the capsule, the device unrolls and adheres to the mucosal membrane (440). The flexible device offers several advantages. Owing to its large size, it offers higher degree of adhesion and decreased interference from other obstacles compared to smaller devices. Further, the flexibility of the device allows it to conform to the surface undulations of the mucosal membrane.

In yet another embodiment, the device includes actuation means to facilitate transport. The actuation means may be one of a variety of means for applying energy to facilitate transport, including but not limited to iontophoresis, osmotic pressure, and mechanical energy sources. In one embodiment, the actuation means include at least one electrode and a battery. FIG. 13 is an illustration of a device that contains an exemplary actuation means. The device contains a mucoadhesive compartment (510) which is proximal to a drug compartment (520). The drug compartment (520) is proximal to an electrode (550) which is in electronic communication with and can be activated by a battery (540). The device also contains a supporting compartment (560), which also includes means to complete the electric circuit. Typically, the supporting compartment is distal to the mucoadhesive component. When the device is placed on a patient's body, the supporting compartment forms the outermost surface of the device.

The different components of the multicompartmental devices are further described below.

a. Supporting Layer

The supporting layer (also referred to herein as a “supporting compartment”) (see e.g., element 110 of FIG. 9 and element 230 of FIG. 10) is formed of a biocompatible, poorly permeable and mechanically strong material. This compartment prevents the entry of enzymes into the device and leakage of drug out of the device (prior to the desired time for drug release). Any synthetic or natural polymer can be used to form the protective compartment. The polymer should be sufficiently stretchable such that when the device swells due to water absorption, the supporting compartment does not fall apart. Stretchability can be modified by incorporation of additives into the polymer.

Representative synthetic polymers that can be used for making the supporting compartment include poly(hydroxy acids) such as poly(lactic acid), poly(glycolic acid), and poly(lactic acid-co-glycolic acid), poly(lactide), poly(glycolide), poly(lactide-co-glycolide), polyanhydrides, polyorthoesters, polyamides, polycarbonates, polyalkylenes such as polyethylene and polypropylene, polyalkylene glycols such as poly(ethylene glycol), polyalkylene oxides such as poly(ethylene oxide), polyalkylene terepthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polystyrene, polyurethanes and co-polymers thereof, derivatized celluloses such as alkyl cellulose, hydroxyalkyl celluloses, cellulose ethers, cellulose esters, nitro celluloses, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulfate sodium salt (jointly referred to herein as “synthetic celluloses”), polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), poly(butyric acid), poly(valeric acid), and poly(lactide-co-caprolactone), copolymers and blends thereof. Examples of non-biodegradable polymers include ethylene vinyl acetate, poly(meth)acrylic acid, polyamides, copolymers and mixtures thereof. Examples of biodegradable polymers include polymers of hydroxy acids such as lactic acid and glycolic acid, and copolymers with PEG, polyanhydrides, poly(ortho)esters, polyurethanes, poly(butyric acid), poly(valeric acid), poly(lactide-co-caprolactone), blends and copolymers thereof.

One or more plasticizers may be added to the supporting compartment to facilitate stretching upon swelling of the device. Representative classes of plasticizers include, but are not limited to, abietates, adipates, alkyl sulfonates, azelates, benzoates, chlorinated paraffins, citrates, energetic plasticizers, epoxides, glycol ethers and their esters, glutarates, hydrocarbon oils, isobutyrates, oleates, pentaerythritol derivatives, phosphates, phthalates, polymeric plasticizers, esters, polybutenes, ricinoleates, sebacates, sulfonamides, tri- and pyromellitates, biphenyl derivatives, calcium stearate, carbon dioxide, difuran diesters, fluorine-containing plasticizers, hydroxybenzoic acid esters, isocyanate adducts, multi-ring aromatic compounds, natural product derivatives, nitriles, siloxane-based plasticizers, tar-based products and thioesters. An exemplary plasticizer is glycerol at a concentration of about 2% w/v.

b. Drug Compartment

The drug compartment (see e.g., element 120 of FIG. 9; element 220 of FIG. 10; and elements 320a, b and c of FIG. 11) carries one or more therapeutic molecules to be delivered into or across the mucosal membrane. The devices described herein contain one or more drug compartments.

Drugs

The drug compartment(s) may contain one or more drugs. The drug is selected based on the disease or disorder to be treated or prevented.

In the preferred embodiment the drug is a protein or peptide. However, a wide range of drugs may be included in the compositions. Drugs contemplated for use in the formulations described herein include, but are not limited to, the following categories and examples of drugs and alternative forms of these drugs such as alternative salt forms, free acid forms, free base forms, and hydrates.

Drug compartment(s) may be prepared using a pharmaceutically acceptable carrier composed of materials that are considered safe and effective and may be administered to an individual without causing undesirable biological side effects or unwanted interactions. Suitable excipients are determined based on a number of factors, including the dosage form, desired release rate of the drug, stability of the drug to be delivered. Excipients include, but are not limited to, polyethylene glycols, humectants, vegetable oils, medium chain mono, di and triglycerides, lecithin, waxes, hydrogenated vegetable oils, colloidal silicon dioxide, polyvinylpyrrolidone (PVP) (“povidone”), celluloses, CARBOPOL® polymers (Lubrizol Advanced Materials, Inc.) (i.e. crosslinked acrylic acid-based polymers), acrylate polymers, other hydrogel forming polymers, plasticizers, crystallization inhibitors, bulk filling agents, solubilizers, bioavailability enhancers and combinations thereof.

c. Mucoadhesive Compartment

The mucoadhesive compartment comprises any suitable, biocompatible mucoadhesive material. In a preferred embodiment, the mucoadhesive compartment contains one or more of Carbopol polymer, pectin and a modified cellulose, such as Carbopol® 934 (Lubrizol Advanced Materials, Inc., pectin (Sigma Chemicals, St. Louis, Mo.), and sodium carboxylmethylcellulose (SCMC, Aldrich, Milwaukee, Wis.). The weight percent of each material in the mixture can be varied to achieve different mucoadhesive effects. In one embodiment, the weight ratio of Carbopol:pectin:SCMC is 1:1:2.

Other suitable mucoadhesive polymers may be used and include, but are not limited to, polyanhydrides, and polymers and copolymers of acrylic acid, methacrylic acid, and their lower alkyl esters, for example polyacrylic acid, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate), carbopol, pectin, chitosan, SCMC, HPMC may also be used.

The mucoadhesive compartment may further comprise a targeting moiety to facilitate targeting of the agent to a specific site in vivo. The targeting moiety may be any moiety that is conventionally used to target an agent to a given in vivo site such as an antibody, a receptor, a ligand, a peptidomimetic agent, an aptamer, a polysaccharide, a drug or a product of phage display.

d. Optional Components

i. Chemical Permeation Enhancers

The device may contain an additional compartment comprising one or more chemical enhancers. In a preferred embodiment, the device includes two or more CPEs, where the CPE's are synergistic enhancer formulations. Preferred synergistic formulations typically contain one or more of the following enhancers: sodium laureth sulfate, decyltrimethyl ammonium bromide, chembetaine, or hexylamine.

The concentration of the one or more CPEs in the device typically has a strong effect on the ability of the CPEs to increase permeability of the drug across a given mucosal surface. The concentration of the CPE is selected to fall within the enhancer's therapeutic concentration window. The therapeutic concentration corresponds with the concentrations at which the enhancer's potency is sufficiently greater than the enhancer's toxicity. Preferably, the concentration of CPE in the device ranges from about 0.01% (w/v) to about 0.1% (w/v).

ii. Means to Prevent Aggregation

In another embodiment, the device will have additional means to prevent aggregation of one device to another device prior to adhesion to the intestinal lumen. Mucoadhesive polymers are very “sticky” and lead to adhesion of devices to each other instead of on the intestinal wall.

Preferably the device has a non-planar shape, such as a hemisphere, which assists in minimizing aggregation of the device. In one embodiment, the devices are modified to as to minimize adhesion, such as by coating the device or the mucoadhesive side with a non-adhesive coating over the mucoadhesive layer or compartment, where the non-adhesive coating dissolves over a short period of time so as to allow the devices to drift away from each other. This non-adhesive coating may be prepared from sugars, polymers, proteins or other molecules.

Alternatively, a multitude of devices may be placed and delivered within a dissolvable container which is under slight over-pressure. Upon dissolution of the container, the over-pressure pushes the devices away from each other, thereby minimizing self-aggregation.

In another embodiment, the device has flanges (710 a, b, c, and d) that fold onto themselves to prevent adhesion of devices to each other (see FIGS. 18A, B, and C). For example, the device may be placed inside a containment, such as a capsule. In the containment (e.g., capsule), the flanges are in the closed position and the mucoadhesive side is shielded from the outside, that is, the mucoadhesive side faces in. When the devices exit the containment, exposure to moisture in the lumen facilitates opening of the flanges and exposes the mucoadhesive side to the epithelium. This way, the devices are adhesive only after they exit the containment.

iii. Means for Delayed Drug Release In another embodiment, the devices contain means to delay the drug release until the device adheres to the intestinal wall. This feature minimizes the likelihood that the drug will be released from the device prior to its attachment to the mucosa.

This delay can be achieved by an additional coating on the outer surface of the device that dissolves slowly with time. This coating may be prepared using any suitable material that dissolves over a time period between one to 60 minutes following swallowing of the oral drug delivery device so as to improve the delivery of drugs. Quick dissolution, i.e. less than 1 minute following swallowing, will lead to disappearance of the coating prior to device adhesion on the intestine. On the other hand, slow dissolution, i.e. greater than 60 minutes following swallowing, may cause an unsuitable delay of the release of drugs from the device.

iv. Hygroscopic Materials

In one embodiment, the devices contain one or more hygroscopic materials. The hygroscopic material is included in the device in an effective amount to absorb excess water, which would otherwise interfere with mucoadhesion, and thereby assist in the adhesion of the devices to a mucosal surface. Excess water interferes with mucoadhesion. Thus, removal of some amount of water from the desired delivery site increases the likelihood of adhesion of the devices on the intestine.

In one preferred embodiment, a multitude of devices are placed in a containment, such as a capsule, and delivered to a patient. Preferably the containment carries a highly hygroscopic material in addition to drug-containing devices.

### III. Methods of Making the Multicompartment Devices

a. Drug Compartment

The drug compartment may be prepared using various methodologies. In one embodiment, the drug is mixed with appropriate excipients and compressed using a hydraulic press. The pressure used during this step can be varied to affect the dissolution time of the device in vivo. Then a hole punch can be used to cut this disk into smaller disks, such as disks with radii of 1-4 mm. In another embodiment, the drug can be deposited into dyes of various sizes and shapes to make compartment of appropriate sizes and shapes.

In another embodiment, such as illustrated in FIG. 15, the drug may be encapsulated in particulates, typically micro- or nanospheres, each of which may act as an independent compartment. There are several processes whereby particulates can be made, including, for example, spray drying, interfacial polymerization, hot melt encapsulation, phase separation encapsulation, spontaneous emulsion, solvent evaporation microencapsulation, solvent removal microencapsulation, coacervation and low temperature microsphere formation.

In spray drying, the core material to be encapsulated (e.g. the drug) is dispersed or dissolved in a solution. Typically, the solution is aqueous and preferably the solution includes a polymer. The solution or dispersion is pumped through a micronizing nozzle driven by a flow of compressed gas, and the resulting aerosol is suspended in a heated cyclone of air, allowing the solvent to evaporate from the microdroplets. The solidified microparticles pass into a second chamber and are trapped in a collection flask.

Interfacial polycondensation is used to microencapsulate a core material in the following manner. One monomer and the core material are dissolved in a solvent. A second monomer is dissolved in a second solvent (typically aqueous) which is immiscible with the first. An emulsion is formed by suspending the first solution through stirring in the second solution. Once the emulsion is stabilized, an initiator is added to the aqueous phase causing interfacial polymerization at the interface of each droplet of emulsion.

In hot melt microencapsulation, the core material (to be encapsulated) is added to molten polymer. This mixture is suspended as molten droplets in a nonsolvent for the polymer (often oil-based) which has been heated to approximately 10° C. above the melting point of the polymer. The emulsion is maintained through vigorous stirring while the nonsolvent bath is quickly cooled below the glass transition of the polymer, causing the molten droplets to solidify and entrap the core material.

In solvent evaporation microencapsulation, the polymer is typically dissolved in a water immiscible organic solvent and the material to be encapsulated is added to the polymer solution as a suspension or solution in an organic solvent. An emulsion is formed by adding this suspension or solution to a beaker of vigorously stirring water (often containing a surface active agent, for example, polyethylene glycol or polyvinyl alcohol, to stabilize the emulsion). The organic solvent is evaporated while continuing to stir. Evaporation results in precipitation of the polymer, forming solid microcapsules containing core material.

The solvent evaporation process can be used to entrap a liquid core material in a polymer or copolymer. The polymer or copolymer is dissolved in a miscible mixture of solvent and non-solvent, at a non-solvent concentration which is immediately below the concentration which would produce phase separation (i.e., cloud point). The liquid core material is added to the solution while agitating to form an emulsion and disperse the material as droplets. Solvent and non-solvent are vaporized, with the solvent being vaporized at a faster rate, causing the polymer or copolymer to phase separate and migrate towards the surface of the core material droplets. This phase-separated solution is then transferred into an agitated volume of non-solvent, causing any remaining dissolved polymer or copolymer to precipitate and extracting any residual solvent from the formed membrane. The result is a microcapsule composed of polymer or copolymer shell with a core of liquid material.

In solvent removal microencapsulation, the polymer is typically dissolved in an oil miscible organic solvent and the material to be encapsulated is added to the polymer solution as a suspension or solution in organic solvent. Surface active agents can be added to improve the dispersion of the material to be encapsulated. An emulsion is formed by adding this suspension or solution to vigorously stirring oil, in which the oil is a non-solvent for the polymer and the polymer/solvent solution is immiscible in the oil. The organic solvent is removed by diffusion into the oil phase while continuing to stir. Solvent removal results in precipitation of the polymer, forming solid microcapsules containing core material.

In phase separation microencapsulation, the material to be encapsulated is dispersed in a polymer solution with stirring. While continually stirring to uniformly suspend the material, a nonsolvent for the polymer is slowly added to the solution to decrease the polymer's solubility. Depending on the solubility of the polymer in the solvent and nonsolvent, the polymer either precipitates or phase separates into a polymer rich and a polymer poor phase. Under proper conditions, the polymer in the polymer rich phase will migrate to the interface with the continuous phase, encapsulating the core material in a droplet with an outer polymer shell.

Spontaneous emulsification involves solidifying emulsified liquid polymer droplets by changing temperature, evaporating solvent, or adding chemical cross-linking agents. The physical and chemical properties of the encapsulant, and the material to be encapsulated, dictates the suitable methods of encapsulation. Factors such as hydrophobicity, molecular weight, chemical stability, and thermal stability affect encapsulation.

Encapsulation procedures for various substances using coacervation techniques have been described in the prior art, for example, in GB-B-929 406; GB-B-929 401; U.S. Pat. Nos. 3,266,987; 4,794,000 and 4,460,563. Coacervation is a process involving separation of colloidal solutions into two or more immiscible liquid layers (Ref. Dowben, R. General Physiology, Harper & Row, New York, 1969, pp. 142-143.). Through the process of coacervation compositions comprised of two or more phases and known as coacervates may be produced. The ingredients that comprise the two phase coacervate system are present in both phases; however, the colloid rich phase has a greater concentration of the components than the colloid poor phase.

In the melt-solvent evaporation method, the polymer is heated to a point of sufficient fluidity to allow ease of manipulation (for example, stirring with a spatula). The temperature required to do this is dependent on the intrinsic properties of the polymer. For example, for crystalline polymers, the temperature will be above the melting point of the polymer. After reaching the desired temperature, the agent to be encapsulated is added to the molten polymer and physically mixed while maintaining the temperature. The molten polymer and agent to be encapsulated are mixed until the mixture reaches the maximum level of homogeneity for that particular system. The mixture is allowed to cool to room temperature and harden. This may result in melting of the agent in the polymer and/or dispersion of the agent in the polymer. The process is easy to scale up since it occurs prior to encapsulation. High shear turbines may be used to stir the dispersion, complemented by gradual addition of the agent into the polymer solution until the loading is achieved. Alternatively the density of the polymer solution may be adjusted to prevent agent from settling during stirring.

b. Methods for Making Mucoadhesive Compartment

The mucoadhesive compartment may be prepared by dissolving a mucoadhesive polymer in an appropriate solvent, for example water, and coated on the drug compartment. The coating can be achieved spraying, jetting or any other reasonable means of uniformly spreading mucoadhesive material on the drug compartment. Alternatively, the mucoadhesive material may be spread in the dry form. In this mode, solid powder of mucoadhesive polymer is placed on the drug compartment and compressed to form a dense, uniform coat.

c. Methods for Making Supporting Compartment

The supporting compartment may be prepared using methods similar to those described above, by replacing the mucoadhesive polymer with a supporting polymer.

### IV. Methods for Selecting One or More CPEs for a Drug Delivery Formulation

To determine which CPEs are best suited for a drug-containing composition, one must first determine the desired site(s) for drug delivery. If local drug delivery within the epithelium is desired, then the preferred CPEs are those that behave primarily via transcellular transport. CPE's that display the most transcellular behavior include cationic and zwitterionic surfactants. Of the transcellular enhancers, the more hydrophobic the CPE, the greater the EP. Thus hydrophobic, transcellular enhancers are typically preferred for local delivery within an epithelial surface.

If systemic drug delivery is desired, then the preferred CPEs are those that behave primarily via paracellular transport. CPE's that display the most paracellular behavior include fatty esters and compounds containing nitrogen-containing rings. Of the paracellular enhancers, the more hydrophobic the CPE, the lower the EP. Thus, hydrophilic paracellelar enhancers are typically preferred for systemic drug delivery.

To determine the concentration for the CPEs for a drug-containing composition, one can use the following method:

1) determine the EP, TP and OP for one or more CPEs at a variety of concentrations

2) use the above information to plot OP versus concentration to determine the initial therapeutic concentration window, and

3) select a concentration within the initial therapeutic concentration window.

Determining the concentration that is therapeutically effective in vivo in humans (and other mammals) involves routine testing. The therapeutic concentration window determined according to the in vitro tests discussed above and described in Example 1, provides a starting point for this determination. However, it is expected that greater concentrations than the therapeutic concentration window determined via in vitro tests will be required in vivo. This increase in concentration for the CPE is likely needed to account for the presence of serum and mucus proteins in vivo, which interact with the CPE and dilute the effective concentration of the CPE when it is administered to a patient.

### V. Uses for Compositions

The compositions described herein may be designed for drug delivery to or through a variety of mucosal surfaces, including intestinal mucosa, buccal mucosa, and vaginal mucosa. In one preferred embodiment, the compositions are designed for drug delivery to the intestinal epithelium or within the intestinal epithelium.

CPEs that are useful for facilitating transepithelial drug transport include CPEs that enter the epithelium primarily using a paracellular transport mechanism. Exemplary CPEs that enter the epithelium primarily using a paracellular transport mechanism include 0.1% w/v phenylpiperazine, 1% w/v methylpiperazine, 0.01% w/v sodium laureth sulfate, 1% w/v menthone, and 0.01% w/v N-lauryl sarcosinate.

CPEs that are useful for facilitating drug transport into epithelial cells are CPEs that enter the epithelium primarily using a transcellular transport mechanism. Formulations containing these CPEs can be useful in treatment or prevention of diseases of the epithelia, including pre-cancerous cervical neoplasia and chronic obstructive pulmonary disease. Exemplary CPEs that enter the epithelium primarily using a transcellular transport mechanism include cationic and zwitterionic surfactants. However, the cationic surfactants possessed the highest MTT-associated toxicity levels of any of the chemical categories. Thus, cationic surfactants are only useful for oral drug delivery compositions when formulated in combination with other enhancers in a synergistic fashion. In contrast, zwitterionic surfactants demonstrated little toxicity to the mitochondria. Therefore, zwitterionic surfactants may be useful CPEs for oral drug delivery formulations designed to deliver drug into epithelial cells.

### EXAMPLES

### Example 1

### Potency and Toxicity for Individual CPEs

Chemical Enhancers

Fifty-one enhancers from 11 distinct chemical categories were chosen for this study. These categories include anionic surfactants (AS), cationic surfactants (CS), zwitterionic surfactants (ZS), nonionic surfactants (NS), bile salts (BS), fatty acids (FA), fatty esters (FE), fatty amines (FM), sodium salts of fatty acids (SS), nitrogen-containing rings (NR), and others (OT). A complete list of enhancers examined in this study is provided above in Table 1. Compounds were selected to reflect a diverse library of enhancers and to include several commonly-studied CPEs. All compounds were tested at concentrations of 1, 0.1, and 0.01% w/v, and were completely soluble in Dulbecco's Modified Eagles Medium (DMEM, American Type Culture Collection (ATCC), Rockville, Md.).

Cell Culture

Caco-2 cell line HTB-37 (ATCC, Rockville, Md.), derived from human colon cells, was used for all experiments. Cells were maintained in DMEM supplemented with 25 IU/ml of penicillin, 25 mg/L of streptomycin, 250 ug/L of amphotericin B and 100 ml/L of fetal bovine serum. Monolayers were grown on BD Biocoat™ collagen filter supports (Discovery Labware, Bedford, Mass.) according to supplier instructions. At the end of the growth period, the integrity of the cell monolayer was confirmed by transepithelial electrical resistance (TEER) measurements (Millicell-ERS voltohmmeter, Millipore, Billerica, Mass.). Only monolayers with TEER values over 700 Ω-cm² were used for further experimentation.

TEER Experiments

Upper filter supports containing viable Caco-2 monolayers were transferred into a 24-well BD Falcon plate and 1 ml of media was dispensed into each basolateral compartment. Solutions containing the CPE (“enhancer solutions”) were applied to the apical compartment and TEER readings were taken at 10 minutes. TEER recovery was assessed by removing enhancer solutions after 30 minutes, applying fresh media, and measuring TEER values at 24 hours.

Calculation of Enhancement Potential (EP)

All TEER values were normalized by their initial values. EP was calculated as the reduction in TEER of a Caco-2 monolayer after 10 minutes of exposure to that CPE, normalized to the reduction in TEER after exposure to the positive control, 1% Triton X-100, using Equation 1.

Methyl Thiazole Tetrazolium (MTT) Experiments

Caco-2 cells were seeded at 10⁵ cells/well onto a 96-well plate. Enhancer solutions (100 μl) were applied for 30 minutes. 10 μl of reagent from an MTT kit (American Type Culture Collection, Rockville, Md.) was applied to each well for 5 hours, after which 100 μl of detergent was applied to each well and allowed to incubate in the dark at room temperature for about 40 hours. Absorbance was read at 570 nm (MTT dye) and 650 nm (detergent). Toxicity potential (TP) values are reported as the fraction of nonviable cells, as compared to the negative control, DMEM. TP values range from 0 to 1, with 0 indicating no mitrochondrial toxicity, and 1 representing maximum toxicity.

Permeability Experiments

Solutions containing CPEs and 1 μCi/ml of tritium-labeled mannitol or 70 kDa dextran (American Radiolabeled Chemicals, St. Louis, Mo.) were applied to the apical side of Caco-2 monolayers. Samples were taken from the basolateral compartment every 10 minutes for 1 hour and the radiolabeled contents were analyzed with a scintillation counter (Packard Tri-Carb 2100 TR, Meriden, Conn.). Permeability was calculated using a standard equation (see P. Karande, et al., J Control Rel., 110:307-313 (2006)):

$$P = \frac{Δ M}{{C}_{M} {A}_{xs} Δ t} \qquad Eq . 4$$

where ΔM is the amount of solute transported across the barrier in the time Δt, C_{M} is the concentration of solute in the apical compartment, and Aₓₛ is the cross-sectional area of epithelium in contact with the apical solution.

Positive control experiments were performed on BD Biocoat™ filter supports in the absence of cells. Exchange of tritium with water was monitored and did not pose an issue for this system.

Results

Enhancement Potential of CPEs:

Using TEER as a surrogate marker for solute permeability, the potency of all CPE formulations was assessed. An inverse relationship between the permeability of polar solutes and TEER has previously been established in the literature (see M. Tomita, et al., J Pharm Sci. 85:608-611 (1996) and E. Fuller, et al., Pharm Res. 24:37-47 (2007)) and was confirmed using a marker molecule, mannitol, which is 180 Da in size. The use of TEER as an alternative measurement for permeability has several advantages, including convenience and a lack of dependence on the size of the solute, thereby ensuring the generality of results.

EP values of the 153 enhancer formulations exhibited significant variations with respect to concentration. The median EP value of all CPEs was 0.20 at a concentration of 0.01% w/v, increasing to 0.43 at 0.1% w/v, and 0.96 at a concentration of 1% w/v.

At each concentration, EP values also exhibited systematic variations with respect to chemical category. For example, fatty esters possessed very little potency at all concentrations. Surfactants displayed more variation with concentration. At low concentrations (0.01%), most ionic surfactants demonstrated significantly higher potency values compared to other categories (P<0.05). The difference in potency between ionic surfactants and other categories decreased at intermediate concentrations (0.1% w/v) and nearly disappeared at the highest concentration of 1% w/v.

For each chemical category, potency increased with increasing concentration. However, the exact dependence varied significantly for each category.

Toxicity Potential of CPEs Based on MTT Assay

Toxicity potential of enhancers showed a distribution that was almost bimodal (below 0.2 or above 0.8), regardless of the concentration. At low concentration (0.01% w/v), about 80% of CPEs exhibited TP<0.2, whereas at high concentration (1% w/v), the same percent of CPEs exhibited TP>0.8. The median TP values at low, intermediate and high concentration were 0.07, 0.14, and 0.94, respectively.

TP values demonstrated a strong dependence on enhancer chemistry. For example, cationic surfactants often demonstrated high toxicity values at all concentrations. At high concentration (1%), many CPEs in addition to surfactants exhibited high TP. Fatty esters demonstrated extremely low toxicity at all concentrations studied.

Relationships Between EP and TP

Having assessed enhancement and toxicity potentials for 51 enhancers (3 concentrations each), the relationship between the two was then evaluated by plotting the EP and TP results for each CPE on a graph (see FIG. 1). As shown in FIG. 1, there are two major clusters of data points; one is in the ‘low EP-low TP’ and the other is in the ‘high EP-high TP’ region. However, many CPEs fall outside these two clusters. Specifically, 15 out of 153 enhancer formulations recorded high EP (i.e., EP>0.50) and low TP (i.e., TP<0.50), demonstrating the existence of a sizable group of CPEs that are relatively potent and safe.

Overall Potential

The overall potential (OP) for each CPE was calculated using Equation 2. The OP value represents the balance of potency and safety of permeation enhancers.

As a group, anionic surfactants at 0.01% concentration displayed the largest OP, followed by zwitterionic surfactants at 0.01%. A list of the top ten single component CPEs, ranked by their OP value, is provided below in Table 3. The list is dominated by nitrogen-containing rings, zwitterionic surfactants, and anionic surfactants, indicating that chemical category has important implications for potent and safe behavior. Further, surfactants at 0.01% concentration appear frequently on this list of best enhancers.

TABLE 3
Safe and Effective CPEs

| | | Conc. | | |
|---|---|---|---|---|
| CPE | Category | (%) | OP | Rank |
| PPZ | NR | 0.1 | 0.86 | 1 |
| PPS | ZS | 0.01 | 0.80 | 2 |
| MPZ | NR | 1 | 0.73 | 3 |
| MPS | ZS | 0.01 | 0.72 | 4 |
| SLS | AS | 0.01 | 0.70 | 5 |
| SLA | AS | 0.01 | 0.59 | 6 |
| PCC | ZS | 0.01 | 0.57 | 7 |
| MTH | OT | 1 | 0.52 | 8 |
| NLS | AS | 0.01 | 0.51 | 9 |
| CL | NR | 1 | 0.48 | 10 |

Therapeutic Concentration Windows for CPEs

Based on the results mentioned above, the impact of concentration on potency and toxicity behaviors was explored more deeply by analyzing select enhancers at 14 discrete concentrations spanning four orders of magnitude. One CPE from each of the 11 chemical categories was chosen for further investigation.

Of the group of CPEs studied, three different potency and toxicity profiles stood out as being the most typical. The first profile is shown in FIG. 2A and represents data for sodium dioxycholate (SDC), a bile salt. In this instance, the EP curve (circles) fell nearly on top of the TP curve (squares), and at all concentrations the utility of SDC in enhancing permeation is accompanied by comparable toxicity. This profile was fairly uncommon, with Triton-X100 serving as the only other example of this behavior among the 11 CPEs studied.

FIG. 2B, on the other hand, demonstrates a more frequently occurring profile. In the case of the sodium salt of oleic acid (SOA), the drop-off for toxicity occurred at a slightly higher concentration than the drop-off for potency. Therefore, a narrow concentration region existed for SOA in which EP values were still quite high while TP values were low. This region is referred to as the “therapeutic concentration window” for an enhancer. Several other enhancers demonstrated similar trends, including phenyl piperazine and pinene oxide.

The last type of common profile was exemplified by the anionic surfactant, sodium laureth sulfate (SLA), in FIG. 2C. In this situation, the distance between EP and TP curves was small at higher concentration but grew larger as concentration decreased until it reached a plateau at low concentration. Thus, the therapeutic concentration window was larger than in FIG. 2B. This behavior was typical for other charged surfactants, including the cationic surfactant, decyltrimethyl ammonium bromide, and the zwitterionic surfactant, palmityldimethyl ammonio propane sulfonate.

FIG. 2D displays overall potential (OP) data for each of the three previously mentioned examples in FIGS. 2A-C. The width of the peak in OP corresponds to the size of an enhancer's therapeutic concentration window. In the case of SDC (squares, small dashed line), OP never ventured appreciably above zero, indicating that there is no therapeutic concentration for this particular enhancer. On the other hand, SOA (diamonds, large dashed line) and SLA (circles, solid line) exhibited pronounced maxima in OP at 0.15% and 0.02%, respectively.

Exploration of Using Phenyl Piperazine (PPZ) as an Enhancer

Phenyl piperazine (PPZ), the most safe and effective enhancer identified as judged by methods used in this example, is a member of the piperazine family. 0.1% PPZ increased the permeability of the hydrophilic marker molecules, mannitol and 70 kDa dextran, more than 14- and 11-fold, respectively. These values were close to the maximum attainable permeability increases achieved by a positive control.

Recovery of cell monolayers after PPZ-induced permeabilization was also assessed. Upon removal of 0.1% PPZ from the cell monolayer, TEER values recovered to 100% of their original value within 24 hours. This serves as an example of the ability of a CPE to increase transport of drug-like molecules across epithelial cells without inducing toxicity.

### Example 2

### Mechanism of Action for Individual CPEs

Selection of Chemical Permeation Enhancers: The same fifty-one enhancers used in Example 1 were tested in Example 2.

Cell Culture:

The same cell culture used in Example 1 was used in Example 2.

TEER Experiments:

The same procedure for TEER experiments described above with respect to Example 1 was used in Example 2.

Calculation of EP:

EP was calculated using Equation 1, as described above in Example 1.

MTT Experiments:

MTT kits were used to determine toxicity as described above in Example 1.

Lactate Dehydrogenase (LDH) Experiments

In addition to the MTT experiments described in Example 1, above, release of LDH from the caco-2 cells was measured as follows. Caco-2 cells were seeded at 10⁴ cells/well onto a 96-well plate. Enhancer solutions (100 μl) were applied for 30 minutes. 25 μl of the solution was then transferred to a fresh 96-well plate and mixed with 25 μl of LDH reagent from the CytoTox 96® assay (Promega, Madison, Wis.) and allowed to react for 30 minutes in the dark at room temperature. Stop solution (25 μl) was then added to each well, and the absorbance was read at 490 nm. LDH potential (LP) values are reported as the fraction of maximal LDH release, as determined by the positive control lysis solution provided with the assay kit (˜1% Triton-X100). LP values lie on a scale of 0 to 1, with 0 representing no LDH release, and 1 indicating maximum LDH release.

Calculation of Molecular Parameters

Chemical permeation enhancer structures were drawn using the program Molecular Modeling Pro (ChemSW) and were relaxed to their lowest energy conformation. All parameters were estimated as described in the software. The octanol-water partition coefficient was taken as the average of the three closest of four independent methods: atom-based Log P, fragment addition Log P, Q Log P, and Morigucchi's method.

Fluorescence Microscopy

A solution containing a permeation enhancer and 0.01% (w/v) calcein dissolved in phosphate buffered saline was applied to Caco-2 cells. After 30 minutes, solutions were removed and replaced with a solution containing only calcein. After 1 hour, samples were washed 3× with phosphate buffered saline and viewed with a Zeiss fluorescence microscope.

Results

Comparison of the MTT and LDH Assays

Two of the most common toxicity assays used to assess the damage caused by an enhancer to epithelium are the LDH and the MTT assays (Motlekar, et al., J Drug Target., 13:573-583 (2005); and Aspenstrom-Fagerlund, et al., Toxicology, 237:12-23 (2007)). The LDH assay measures the amount of lactate dehydrogenase enzyme, present in the cytosol, which leaks out of the cell and into the extracellular fluid. In essence, this assay measures the permeability of the cellular membrane to a 144 kDa enzyme. The MTT assay measures the ability of the cell mitochondria to cleave the MTT salt into a formazan product, which accumulates inside of the cell. Therefore, the MTT assay is a good measure of the overall health of the cell, as it indicates the viability of the cell's primary energy-generating organelle. Additionally, it has been shown to be the more sensitive of the two assays (G. Fotakis & J. A. Timbrell, Toxicol Let, 160:171-177 (2006)). Based on these differences, the MTT assay was selected to calculate the quantitative parameter, toxicity potential (TP), of the enhancers.

Generally, the use of the MTT assay in place of the LDH assay to determine TP did not have significant implications for most enhancers, given that the results of the MTT and LDH assays usually correlated very well. Only a small percentage (14%) of the CPEs tested did not show a strong correlation between the MTT and LDH assays. Most prominently, zwitterionic surfactants tended to display high LP values but low TP values. Thus, although zwitterionic surfactants are effective in perturbing the membrane of epithelial cells (thereby causing LDH to leak out of the cells), they do not induce toxicity to the mitochondria.

Discrepancies in the toxicity information gathered via MTT and LDH assays can be used to reveal the mechanistic nature of the absorption enhancers.

Mechanisms of Enhancer Action—Transcellular and Paracellular Contributions

Enhancement potential can also be determined based on the transcellular and paracellular contributions to permeability, using Equation 5 below:

$$EP = LP + \frac{{E}_{p}}{{E}_{o}^{max}} \qquad Eq . 5$$

where EP is enhancement potential, LP is LDH potential, and

$$\frac{{E}_{p}}{{E}_{o}^{max}}$$

is a term representing paracellular contributions to permeability. Equation 5 states that the overall potency of an enhancer is equal to a transcellular effect plus a paracellular effect.

Equation 5 was used to assess the relative contribution of transcellular and paracellular pathways to permeability of the intestinal epithelium. FIG. 3 shows a plot of EP vs. LP for all enhancers at the various concentrations tested in this example. According to Equation 4, the line EP=LP corresponds to enhancers that act predominantly by the transcellular route (paracellular contributions are negligible). Enhancers lying on the vertical EP axis primarily utilize the paracellular pathway, since there is no relationship between EP and LP when transcellular contributions are negligible. The relative contribution of the paracellular pathway is higher for enhancers falling closer to the EP axis than to the EP=LP line.

Based on the departure of points from EP=LP, it is possible to quantify the extent of contribution of the paracellular pathway to overall enhancement. For this purpose, the parameter

$$K = \frac{( EP - LP )}{EP} ,$$

which represents the relative contribution of the paracellular pathway, can be calculated. K values were determined for all enhancers, with theoretical values ranging from 0 (predominantly transcellular) to 1 (predominantly paracellular).

For example, 1% EDTA (EP=0.98, LP=0.27) yields K=0.72, indicating that it enhances in vitro transport primarily due to contributions from the paracellular pathway, a conclusion that is consistent with the literature (Hess, et al., Eur J Pharm Sci, 25:307-312 (2005)).

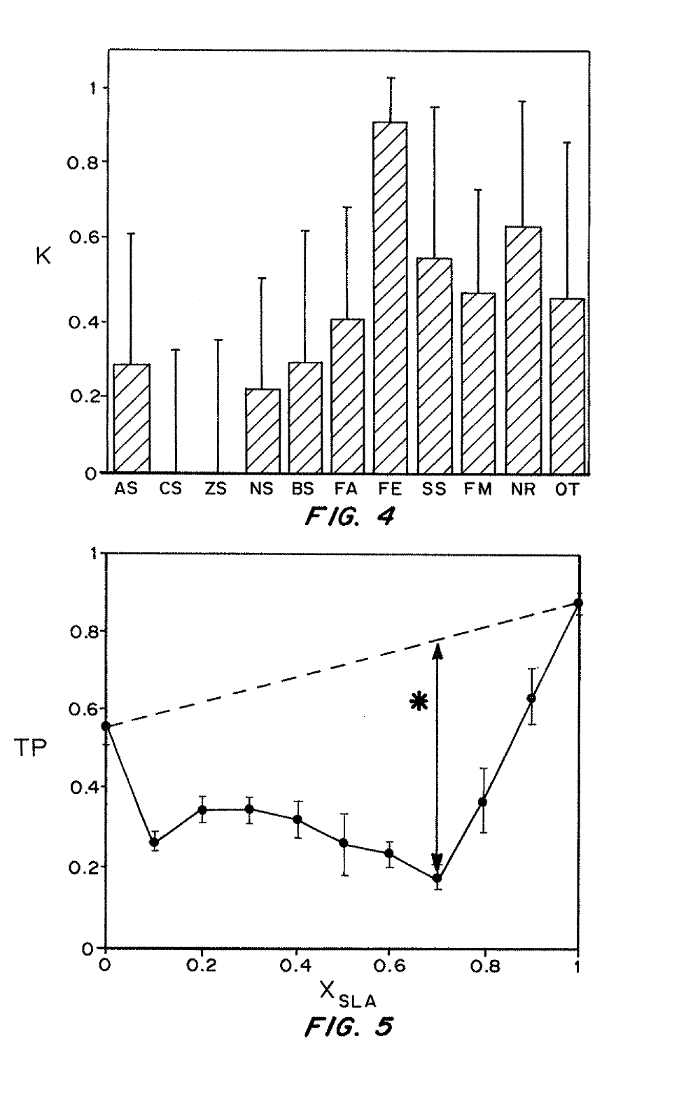

Analysis of enhancer categories based on K is shown in FIG. 4.

Although K values can vary significantly within the same category, these data provide a general idea of the mechanistic behavior of each chemical group. As a whole, fatty esters (FE) displayed by far the most paracellular behavior, followed by nitrogen-containing rings (NR). Cationic (CS) and zwitterionic (ZS) surfactants demonstrated the most transcellular behavior. These surfactants are known to disrupt membrane structure (see E. S. Swenson & W. Curatolo, Adv Drug Deliv Rev, 8:39-92 (1992)).

In general, the route of enhancement (transcellular vs. paracellular) was not dramatically altered by a change in enhancer concentration, from 0.01% to 0.1% w/v or 0.1% to 1% w/v. About half of the time, the change in K values was less than 0.1; and in 83% cases, the change in K values was less than 0.5. Larger changes in K were less prominent. Notable exceptions to this trend include all 5 of the anionic surfactants examined, which become increasingly paracellular as concentration was decreased.

### Molecular Origins of Mechanism of Action

In order to gain insight into the molecular features of a chemical permeation enhancer that affect potency, 22 molecular descriptors, including the octanol-water partition coefficient (Log P), components of solubility parameters (dispersive, polar and hydrogen bonding), and polar surface area were calculated for each enhancer. These parameters were reduced to a set of eight independent variables by assessing their correlation coefficients. These eight parameters were then analyzed for correlations with potency (EP). The data set at 0.01% concentration was chosen for analysis because it had the greatest distribution of EP values, and thus the greatest potential to reveal trends.

Of all of the molecular descriptors that had been calculated, the Log P of the enhancers showed most notable correlations with EP. Specifically, two distinct trends were observed when EP was plotted versus Log P. The first trend demonstrates a direct correlation between the two (r²=0.9). 83% of permeation enhancers in this region are transcellular in nature (i.e., K<0.5). The other trend, shows an inverse trend between EP and Log P (r²=0.77). 96% of enhancers in this region are paracellular (i.e., K>0.5). The analysis of a graph of Log P versus EP thus reveals two separate trends for enhancers acting through transcellular or paracellular routes. First, the potency of transcellular enhancers scales directly with enhancer hydrophobicity; and second, the potency of paracellular enhancers scales inversely with hydrophobicity.

Applications of Chemical Permeation Enhancers in Intraepithelial Drug Delivery

The zwitterionic surfactant 0.01% (w/v) palmityldimethyl ammonio propane sulfonate (PPS) was chosen for intraepithelial studies, as it was shown to be safe and effective while utilizing the transcellular route in vitro (EP=0.8, TP=0, K=0).

0.01% PPS permeabilized epithelial cells and allowed the entry of the marker molecule, calcein, into the epithelial cells. While the negative control was only able to deliver calcein in between the cells, 0.01% PPS enabled the transport of calcein into more than 75% of epithelial cells.

In order to confirm that this permeabilization was due to a potent transcellular mechanism, the experiment was also performed with 0.1% phenylpiperazine, a safe and effective paracellular enhancer (EP=0.95, TP=0.09, K=0.86). Use of phenylpiperazine resulted in a situation similar to the negative control, indicating that intraepithelial delivery can be achieved only through transcellular means.

It was also confirmed that 0.01% PPS did not damage cell monolayer structure through TEER recovery experiments.

### Example 3

### Combinations of CPEs

Generation of Chemical Permeation Enhancer Library

A large number of combination CPE formulations were screened in order to understand the enhancer interactions affecting synergy. All single enhancers used to build mixture formulations in this study had previously been shown to possess relatively high potency and high toxicity within their chemical category. Because these single enhancers were already extremely potent, the focus was to reduce values of the toxicity potential (TP).

One enhancer was selected from each of 11 distinct chemical categories listed in Table 1. Each enhancer selected possessed high single component toxicity relative to other enhancers in that chemical category. For the binary study, each enhancer was paired with every other enhancer, for a total of 55 pairs. Each pair was tested at total concentrations of 0.1% and 1% (w/v) and at 11 weight fractions varying from 0 to 1, with a step size of 0.1. A total of 1,210 binary test formulations were generated.

The top 25 combinations (based on synergy values) were then analyzed for potency, which enabled the assessment of the overall potential (OP) of the formulation. Promising formulations were evaluated for usefulness in transepithelial enhancement applications.

The synergy results obtained from binary analysis were used to generate an enhancer library for the investigation of ternary formulations, performed in the same fashion. A ternary library was generated from four enhancers with the best performance from the binary study. Ternary combinations were only studied at 0.1% (w/v). A total of 264 ternary formulations were analyzed.

Enhancers were completely soluble in DMEM, which was used as the solvent.

Cell Culture:

Cell Cultures were prepared as described above with respect to Example 1, with the following exception. Monolayers were grown on BD Biocoat™ collagen filter supports (Discovery Labware, Bedford, Mass.) according to supplier instructions, with the following exception: 10% FBS was used to supplement the basal seeding medium provided by the supplier.

TEER Experiments:

The same procedure for TEER experiments described above with respect to Example 1 was used in Example 3.

Calculation of EP:

EP was calculated using Equation 1, as described above in Example 1.

MTT Experiments:

MTT kits were used to determine toxicity as described above in Example 1.

Permeability Experiments:

The same procedure for permeability experiments described above with respect to Example 1 was used in Example 3. Water-tritium exchange was monitored and did not pose a problem for this system.

Results

MTT Screening and Synergy Calculation

Over 1200 binary combinations and 264 ternary combinations were tested for toxicity using the MTT assay. The synergy for each combination of CPEs was calculated using Equation 3.

A graphical representation of synergy in a binary system, containing decyltrimethyl ammonium bromide (DTAB) and sodium laureth sulfate (SLA), is shown in FIG. 5.

At 0.1% total concentration, pure decyltrimethyl ammonium bromide (DTAB), located at X_{SLA}=0, and pure sodium laureth sulfate (SLA), located at X_{SLA}=1, possessed high TP values of 0.56 and 0.88, respectively. If no synergy existed between these two components as their weight fractions were varied, then the TP values of the mixtures would fall along the dashed line. However, all combinations of DTAB and SLA possessed experimental TP values well below the dashed line. The magnitude of the synergy is the difference between the experimental value and the expected value. The maximum value of synergy achieved for the SLA-DTAB system was 0.61 and occurs at X_{SLA}=0.7.

Distribution of TP and Synergy

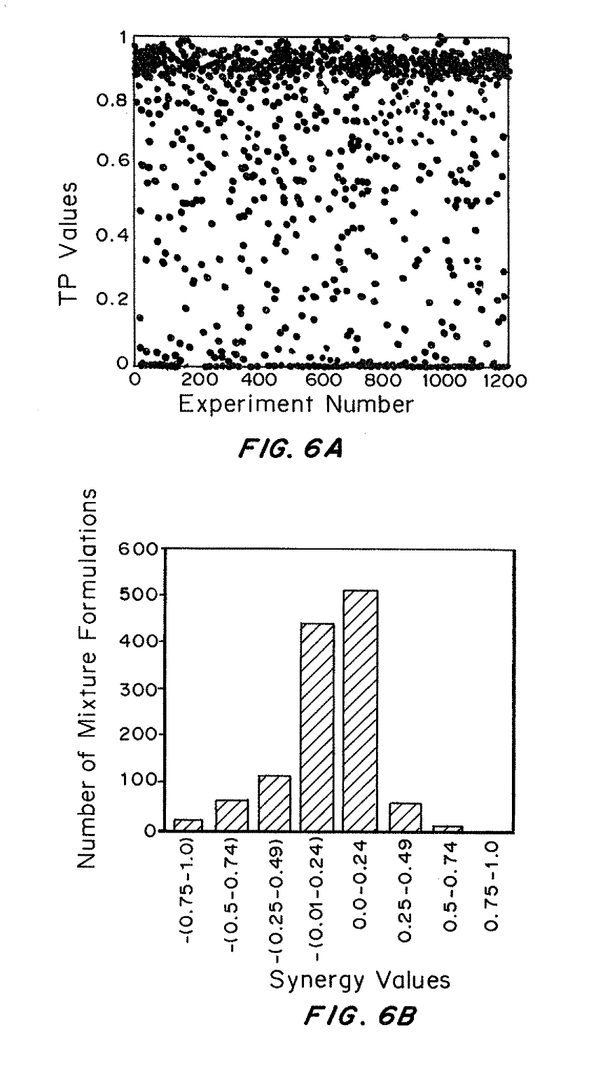

FIG. 6A shows the distribution of TP values for all of the binary enhancer combinations tested in this experiment. The majority of mixture formulations displayed relatively high toxicity (TP>0.8). This is because the single enhancers selected to form combinations possessed high toxicities on their own and because synergy did not occur frequently. As demonstrated in FIG. 6B, most binary mixtures did not display marked synergistic behavior, with 79% of mixtures possessing a synergy value between −0.25 and 0.25. Although most enhancer mixtures demonstrated low or negative synergy, a small but significant fraction (6%) was comprised of synergistic enhancer formulations (SEFs), i.e. Synergy greater than 0.25 (S>0.25).

Potency Analysis

The top 25 binary SEFs (selected based on synergy values) were analyzed for potency. Enhancement potential (EP) was used as a quantitative measure of potency, with an EP value of 1 representing maximum enhancement. FIG. 7A shows the EP and TP values of the 25 most synergistic binary combinations. As noted above in Example 1, single enhancers often exhibited undesirable behavior in the form of either low potency or high toxicity. None of the single enhancers possessed both high EP and low TP values, a requirement for enhancer candidates. On the other hand, all of the top 25 enhancer combinations possessed both high EP and low TP values, with EP>0.6 and TP<0.5, indicating that they are both potent and relatively non-cytotoxic.

The parameter, overall potential (OP), enables an effective comparison of enhancers by quantifying the difference between potency and toxicity of the mixture. Synergistic enhancer combinations were capable of producing formulations with much higher OP values compared to single permeation enhancers. FIG. 7B provides the OP values for the top 25 binary SEFs identified in this Example. A significant number of SEFs possessed very high OP values. For example, binary analysis identified 10 combinations with OP≧0.80, compared to two formulations with OP≧0.80 from the single enhancer study disclosed in Example 1.

Certain CPEs appeared to be particularly prolific in the generation of SEFs. These enhancers, namely, sodium laureth sulfate (SLA), decyltrimethyl ammonium bromide (DTAB), chembetaine (CBC), and hexylamine (HAM), were about 4-5 times more likely to produce an SEF than the other CPEs of the binary study.

Ternary Enhancer Combinations

Four enhancers, sodium laureth sulfate (SLA), decyltrimethyl ammonium bromide (DTAB), chembetaine (CBC), and hexylamine (HAM), were tested further for their ability to produce synergistic behavior through ternary combinations. Ternary formulations were only tested at 0.1% (w/v) total concentration because 97% of SEFs from the binary study occurred at this lower concentration.

37% of ternary combinations tested resulted in an SEF (i.e., S>0.25), compared with 6% of binary formulations. A typical example of the synergy achieved with ternary mixtures can be found in the combination of hexylamine (HAM), sodium laureth sulfate (SLA), and decyltrimethyl ammonium bromide (DTAB) at a total concentration of 0.1% (w/v). Although the individual pure components tested in Example 1 were relatively toxic to Caco-2 cells, much that toxicity was significantly reduced when these enhancers were used in combination. The maximum synergy value obtained by this mixture was 0.67, which occurred at X_{HAM}=0.1, X_{SLA}=0.6 and X_{DTAB}=0.4.

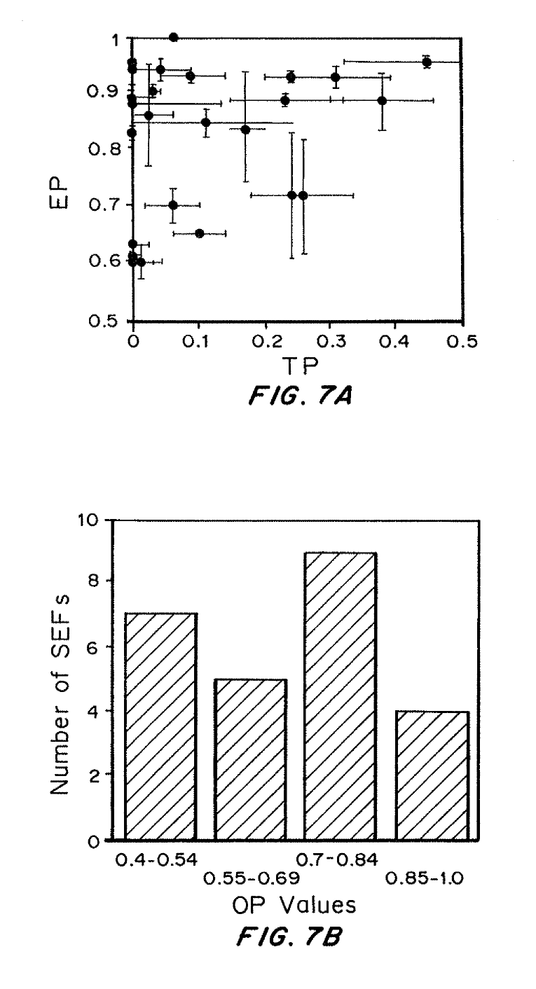

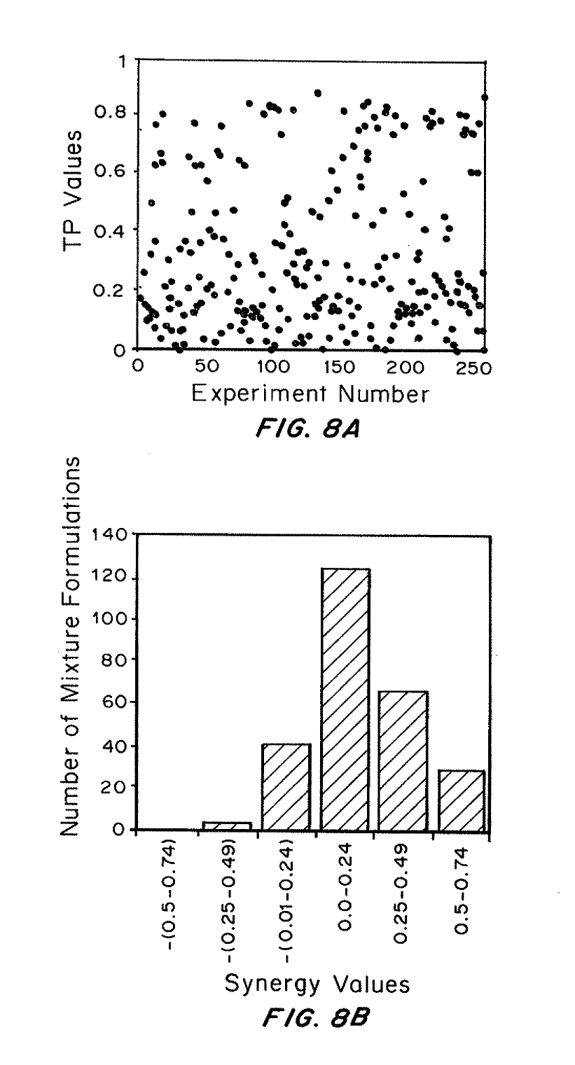

FIGS. 8A and B demonstrate the marked improvement in the ability to identify toxicity-related synergy when thoughtfully selecting enhancers for ternary formulations. TP values for each of the 264 ternary mixtures are plotted in FIG. 8A. When compared with FIG. 6A, it can be seen that the average TP value achieved by the ternary study, 0.32, was much lower than that obtained by the binary study, 0.69. Additionally, a significant shift is observed in the distribution of synergy values (FIG. 8B). A majority of synergy values was positive in the case of ternary formulations, compared to the broad distribution achieved by the binary investigation (FIG. 6B).

The top 15 SEFs identified by ternary analysis were further investigated for their potency via TEER experiments. All EP values fell above 0.9, indicating that these top SEFs were extremely potent.

Overall potential (OP) values were calculated. 6% of ternary mixtures possessed OP values greater than 0.75, compared to 1% of both single and binary formulations. Approximately 3% of all ternary combinations achieved OP values above 0.9, which indicates high potential for use in drug delivery formulations. In contrast, no single enhancer and only 0.3% of binary formulations met such criterion. These results underscore the ability to efficiently obtain higher synergy values, and therefore better enhancer candidates, when moving to ternary formulations.

Transepithelial Drug Delivery

Several of the leading SEFs with the highest OP values were evaluated for their ability to increase the transepithelial permeability of two model drug compounds, mannitol (MW=182 Da) and dextran (MW=70 kDa). The average permeability values for mannitol and dextran in the absence of CPEs are 4.3×10⁻⁷±2.3×10⁻⁷ and 4.9×10⁻⁷±2.3×10⁻⁷, respectively. The permeability of these molecules increased significantly in the presence of the SEFs 0.1% HAM-SLA (X_{HAM}=0.6 and X_{SLA}=0.4) and 0.1% SLA-DTAB-CBC (X_{SLA}=0.5, X_{DTAB}=0.3, and X_{CBC}=0.2). Both SEFs are capable of high permeation increases, 15- and 9-fold for mannitol and dextran, respectively.

### Example 4

### CPEs as Microbicides

Minimum Inhibitory Concentration (MIC) Estimation in B. thailendensis

Minimum inhibitory concentration against B. thailendensis was determined. Broth microdilution method was followed for MIC determination. Briefly, fresh cultures were grown on the day of experiment using the protocol described below.

Bacterial Strains, Growth Media and Culture Conditions

Wild-type E. coli (strain ER2738) was purchased from New England Biolabs (Ipswich, Mass.) and was used as the model gram negative pathogen. Leuria-Bertani (LB) broth (10 g tryptone 1-1, 5 g yeast extract 1-1, 10 g NaCl 1-1) made in ultrapure water and sterilized via autoclaving (121° C., 15 min) was used for culturing E. coli. All components for making the LB broth were purchased from Fisher Scientific (Fairlawn, N.J.). Precultures were prepared for each experiment by streaking stock solution (frozen in cryovials at −80° C.) on LB agar plate. After overnight incubation of the plates at 37° C., one colony was picked and loop-inoculated into a culture tube containing 5 ml LB broth. The culture tube was incubated 15-18 h at 37° C. on a rotary shaker at 250 rpm. At the end of incubation period, one hundred micro-liters of this culture was transferred into a new culture tube containing 5 ml LB broth and grown to an OD600 value of 0.5 under the same incubation conditions. The OD600 cultures were diluted by a factor of 103 in LB broth as working concentration and used immediately to minimize change in bacterial count.

Low sodium Leuria-Bertani (LSLB) broth (10 g tryptone 1-1, 5 g yeast extract 1-1, 5 g NaCl 1-1) made in ultrapure water and sterilized via autoclaving (121° C., 15 min) was used for culturing B. thailendensis. Culturing protocol was same as given above for E. coli.

The cultures were adjusted to 5.5×10⁵ cfu/ml and used within 30 minutes to minimize change in bacterial counts. Cultures were dispensed in 96-well cell culture polypropylene plates (Corning, Lowell, Mass.) at 90 μl/well. Serial dilutions of test formulations were made at 10× concentration. Inoculums in each well were incubated with 10 μl of test formulation dilutions for 18 hours at 37° C. under humidified conditions. At the end of incubation period, the plates were visibly inspected for bacterial growth. Colonies were counted for selected wells by plating culture dilutions on LSLB plates.

Keratinocyte Cell Culture

Primary epidermal keratinocyte cultures from an adult human source (HEKa) were purchased from Invitrogen Corp (Carlsbad, Calif.) and used for all cytotoxicity experiments. Cells were maintained in a humidified incubator (37° C., 5% CO₂), in EpiLife medium with 60 μM calcium and phenol red, supplemented with 10 ml/l human keratinocyte growth supplement, 5 IU/ml penicillin and 5 μg/ml streptomycin. All components of growth media were purchased from Invitrogen Corp (Carlsbad, Calif.). Cells were grown to 70-80% confluence in cell culture flasks (Corning, Lowell, Mass.) as per suppliers' protocols.

Screening for Cytotoxicity

At the end of the growth period, keratinocyte cells were seeded at a density of 10⁴ cells/well in 96-well tissue culture treated polystyrene plates (Corning, Lowell, Mass.) and incubated overnight to allow cell attachment. Cells were supplied with fresh EpiLife medium (90 μl/well) at the start of experiment, followed by application of test formulations (10 μl/well). The final concentration of test formulations in each well was 0.0001% w/v. This concentration limit was determined based on the LC₅₀ values of component chemicals for HEKa cell line, which were determined in a separate experiment. The cells were incubated with the test formulations for 1 hour. At the end of the incubation period, culture media was aspirated and replaced with 100 μl of EpiLife medium without phenol red. Ten microliters of methyl thiazole tetrazolium solution (5 mg/ml) in phosphate buffered saline was applied to each well for 4 hours, after which 100 μl of acidified sodium lauryl sulfate solution (10% w/v in 0.01 N hydrochloric acid) was added to each well. The plates were incubated for 16 hours in a humidified environment and absorbance was read at 570 nm.

S20 exhibited high cell viability (high LC₅₀) but low antibacterial potency. BZK, on the other hand, exhibited high antibacterial potency but low cell viability (low LC50). Mixtures of BZK:S20 in the range of 30-70% BZK exhibited the ideal behavior. These formulations were tested for stability and potency against B. thailandensis. BZK exhibited low MIC (0.00048% w/v) and LC₅₀ (0.00078% w/v), whereas S20 exhibited negligible toxicity and potency in the range of concentrations studied. Binary compositions of BZK:S20 exhibited higher LC₅₀ values compared to BZK alone, indicating that addition of S20 to BZK decreases toxicity. However, addition of S20 also led to decreased potency as judged by increased MIC values.

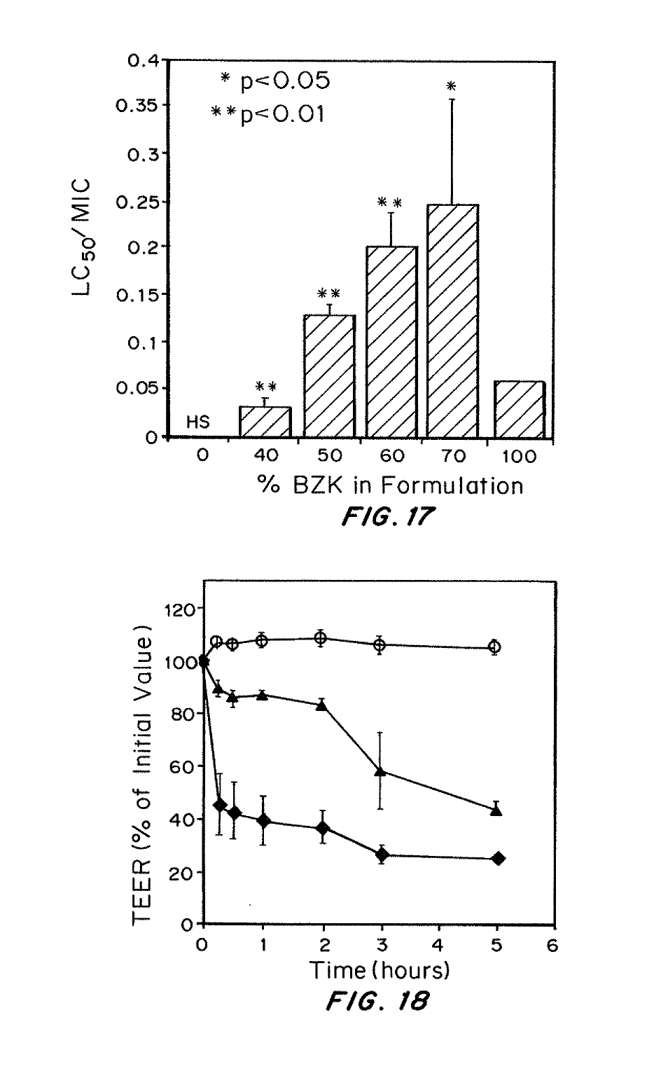

With two independent parameters (MIC and LC₅₀), it is difficult to determine the benefits offered by binary formulations compared to single surfactant formulations. Therefore, the ratio of these two quantities (LC₅₀/MIC) was used for determining the benefits of these formulations as potential microbicide (FIG. 17). The LC₅₀/MIC ratios revealed that formulations of BZK and S20 exhibit up to 3-fold higher LC₅₀/MIC ratio compared to BZK alone. Also, the LC₅₀ values for all three formulations were higher than those of BZK (p<0.05), demonstrating their advantage as microbicides over application of BZK alone.

### Example 5

### Testing CPEs In Vitro and In Vivo

PPS as a Model Permeation Enhancer

To demonstrate the efficacy of CPEs in enhancing transport of therapeutic macromolecules, a zwitterionic surfactant Palmityldimethyl ammonio propane sulfonate (PPS) was chosen as described in Example 2. PPS was chosen based on its overall potential as described in Table 3 (Example 1), and its efficacy in enhancing transport of various markers including mannitol, dextran, and calcein at a low concentration of 0.01% w/v (Examples 1 and 2). PPS was tested at concentrations of 0.01 and 0.03% w/v for cell culture; and 0.1 and 1% w/v for in vivo drug absorption studies. PPS was completely soluble in respective vehicles (DMEM cell culture media for cell culture studies, and sterile saline (0.9% NaCl w/v) for in vivo studies).

Cell Culture

Caco-2 cell line HTB-37 (ATCC, Rockville, Md.), derived from human colon cells, was used for all experiments. Cells were maintained in DMEM supplemented with 25 IU/ml of penicillin, 25 mg/L of streptomycin, 250 μg/L of amphotericin B and 100 ml/L of fetal bovine serum. Monolayers were grown on BD Biocoat™ HTS collagen filter supports (BD Biosciences, Bedford, Mass.) according to supplier instructions. At the end of the growth period, the integrity of the cell monolayer was confirmed by transepithelial electrical resistance (TEER) measurements (Millicell-ERS voltohmmeter, Millipore, Billerica, Mass.). Only monolayers with TEER values in the range of 150-200 Ω-cm² were used for further experimentation.

TEER Experiments

Upper filter supports containing viable Caco-2 monolayers were transferred into a 24-well BD Falcon plate, and 1.4 ml of media was dispensed in each basolateral compartment. Solutions containing PPS were applied to the apical compartment and TEER readings were taken at regular intervals up to 5 hours.

Transwell Permeability Experiments

Solutions containing PPS (0.01 or 0.03% w/v) and tracer molecules (sulforhodamine-B or FITC-insulin; 0.15 mg/well) were applied to the apical side of Caco-2 monolayers, and the plates were incubated for 5 hours with gentle shaking. At regular time intervals, 100 μl of sample was withdrawn from basolateral chamber to quantify the amount of sulforhodamine-B/FITC-insulin transported across the monolayer. The withdrawn sample was immediately replaced with an equivalent amount of the experimental media. Withdrawn samples were analyzed using a Tecan Saffire™ fluorescent microplate reader (Tecan Group Ltd, Mannedorf, Switzerland) at respective wavelengths for FITC-insulin (Ex 488 nm; Em 525 nm) and sulforhodamine-B (Ex 560 nm and Em 590 nm).

Permeability and Enhancement Ratio Calculation

Permeability (P) was calculated by using a standard equation as described in Example 1. Transport enhancement ratios were calculated according to following equation as described by Thanou et al. (see Thanou et al., J Pharm Sci., 90(1):38-46 (2001)):

$$ER = \frac{{P}_{app - Enhancer}}{{P}_{app - Control}}$$

TEER Reversibility Experiments

To ensure the permeation enhancement effect of PPS, TEER recovery was assessed by removing PPS solution (0.03% w/v) after (i) 10 min, and (ii) 1 hour, applying fresh media, and measuring the TEER following incubation at 37° C. for 24 hours.

Methyl Thiazole Tetrazolium (MTT) Experiments

Toxicity of PPS was tested on Caco-2 cell line (HTB-37) using an MTT assay. Caco-2 cells were seeded in a 96 well plate at a density of 5×10⁴ cells per well in 96-well plate. 100 μL of PPS solutions (concentrations between 0.0005% and 0.03% w/v) were added, and the plates were incubated at 37° C. for different time points (10 min, 1 hr, and 5 hrs). 10 μL of MTT solution (5 mg/mL) was added to each well for 4 h (37° C.), after which 100 μL of 100% DMSO was applied and the plates were incubated for 1 hr with moderate shaking Absorbance was measured at 570 nm using a Tecan Saffire™ fluorescent microplate reader (Tecan Group Ltd, Mannedorf, Switzerland).

Confocal Laser Microscopy Experiments

Permeation enhancement effect of PPS on FITC-insulin was assessed by confocal microscopy. PPS solution (0.01 or 0.03% w/v) with FITC-insulin (0.15 mg/well) was applied to Caco-2 monolayer; and was incubated for 5 hours (37° C.), after which the solution was removed and the monolayers were fixed overnight with 4% paraformaldehyde (4° C.). Following fixation, cells were gently washed with HBSS, membranes gently removed from the plastic insert, and were mounted on a microscopy slide with DAPI containing cell mounting media (Vectashield® Hardset®, Vector Laboratories, Burlingame, Calif.). All samples were imaged on a confocal microscope (Leica and Olympus Fluoview 500). To quantify the permeation enhancement effect of PPS, confocal images were analyzed using ImageJ image processing software.

In Vivo Experiments with Adult Male Sprague-Dawley (SD) Rats

In vivo efficacy of PPS in enhancing peptide transport was assessed by determining its effect on intestinal transport of salmon calcitonin (sCT, Anaspec Inc, Fremont, Calif.), a poorly permeable therapeutic peptide. Adult male Sprague-Dawley (SD) rats of 275-300 g, fasted for 7 hours were used for all studies. A midline abdominal incision of 2.5-3.0 cm was made in animals anesthetized by 1.5-3.0% isoflurane to expose the gastrointestinal system. 0.5 ml PPS solution (0.1 or 1% w/v in sterile saline) with sCT (3 mg/kg) was injected into the small intestine (duodenum region). Incision was then closed with sterile vicryl seizures (muscular), and Vetbond® tissue glue (skin). Blood samples were collected up to 5 hours by tail vein bleeding (heparinized tubes for plasma Calcium, and EDTA tubes for plasma sCT determination), and plasma was separated for analysis of both pharmacodynamic and pharmacokinetic response. Pharmacodynamic response was measured by quantifying plasma calcium levels using a colorimetric calcium assay kit (Sciencell Laboratories, Carlsbad, Calif.); and pharmacokinetic efficacy was tested by quantifying plasma concentrations of salmon calcitonin (sCT) using an extraction-free ELISA kit (Bachem Americas Inc., Torrance, Calif.) following manufacturer's protocols.

Tissue Histology Experiments

Histological studies were performed to evaluate the possibility of damage caused by PPS in the intestine. 0.5 ml PPS solution (0.1% w/v or 1% w/v) was administered into the intestine using the procedures described earlier, and the animals were euthanized after 5 hrs. The intestine exposed to PPS was excised, fixed in formalin, and was sectioned perpendicularly as 5 μm sections. The sections were stained using hematoxylin-eosin staining and imaged at 50× magnification using an inverted light microscope to determine signs of pathological changes.

Results

Permeability Enhancement Potential of PPS

Permeability enhancement efficacy of PPS was assessed with sulforhodamine-B (558 da) and FITC-insulin (˜6,000 da) on Caco-2 monolayers. At the same time, TEER measurements were used as a surrogate marker for membrane permeabilization efficacy of PPS so as to negotiate for lack of dependence on solute size.

PPS demonstrated concentration dependent decrease in TEER values with 50% (of initial value) with 0.01% w/v, and almost 70% (of initial value) with 0.03% w/v at 5 hours. However, the higher concentration demonstrated a very rapid drop in TEER (−50% in 15 minutes) suggesting a prompt mode of action for PPS (FIG. 18).

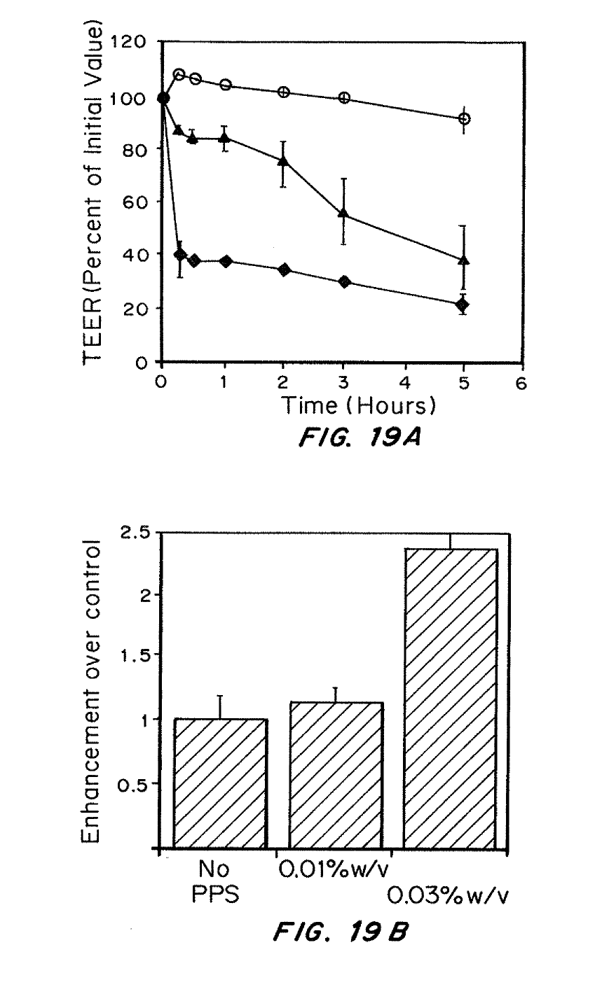

Intraepithelial transport of sulforhodamine-B and FITC-insulin also confirmed the TEER observations. With application of PPS, sulforhodamine transport increased in a concentration dependent manner resulting in 1.7 fold (0.01%), and 2.6 fold (0.03%) increase in its transport over negative control (sulforhodamine with no PPS). The FITC-insulin transport however did not increase significantly with lower concentrations of PPS, whereas a 2.3 fold enhancement was seen in FITC-insulin transport following application of 0.03% w/v PPS (FIGS. 19A and 19B). Permeability values are depicted in Table 4.

TABLE 4
Permeability values
under various conditions tested in Example 5

| | | Apparent | |
|---|---|---|---|
| | PPS | Permeability | % |
| Molecule | (%) | (Pₐₚₚ), 10⁻⁶ cm/s | Transport |
| Sulforhodamine-B | — | 3.5 ± 2.3 | 1.9 ± 1.3 |
| (0.15 mg) | 0.01 | 6.0 ± 0.6 | 3.3 ± 0.4 |
| | 0.03 | 9.1 ± 1.6 | 5.1 ± 0.9 |
| FITC-insulin | — | 6.6 ± 1.2 | 3.7 ± 0.7 |
| (0.15 mg) | 0.01 | 7.6 ± 0.7 | 4.3 ± 0.4 |
| | 0.03 | 15.6 ± 0.7 | 8.7 ± 0.4 |

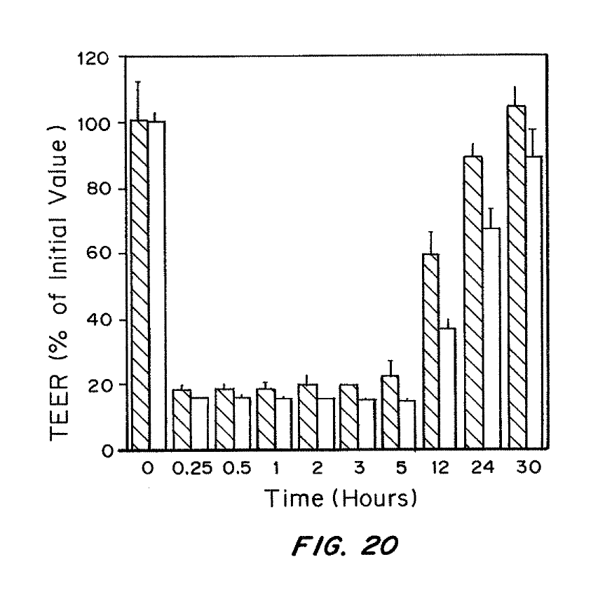

Recovery of cell monolayers after PPS induced membrane permeabilization was assessed by TEER recovery. Caco-2 monolayers were exposed to 0.03% w/v PPS in the apical chamber for different time periods. Following PPS removal, monolayers were incubated for 24 hrs at 37° C., and TEER values were measured at different time-points to assess time-dependent reversibility of TEER. Data represent mean±SD (n=3). As depicted in FIG. 20, TEER values returned to almost 90-100% within 24 hours of removal of PPS following variable exposure times (10 minutes, and 1 hour), with a more rapid recovery seen with shorter exposure time. The speedy recovery of TEER values suggests toward PPS being a safe permeation enhancer for drug molecules without inducing toxicity following limited exposure. TEER data in conjunction with the monolayer transport data suggest that PPS can significantly enhance peptide (FITC-insulin) transport following exposure for a short time.

Toxicity of PPS Based on MTT Assay

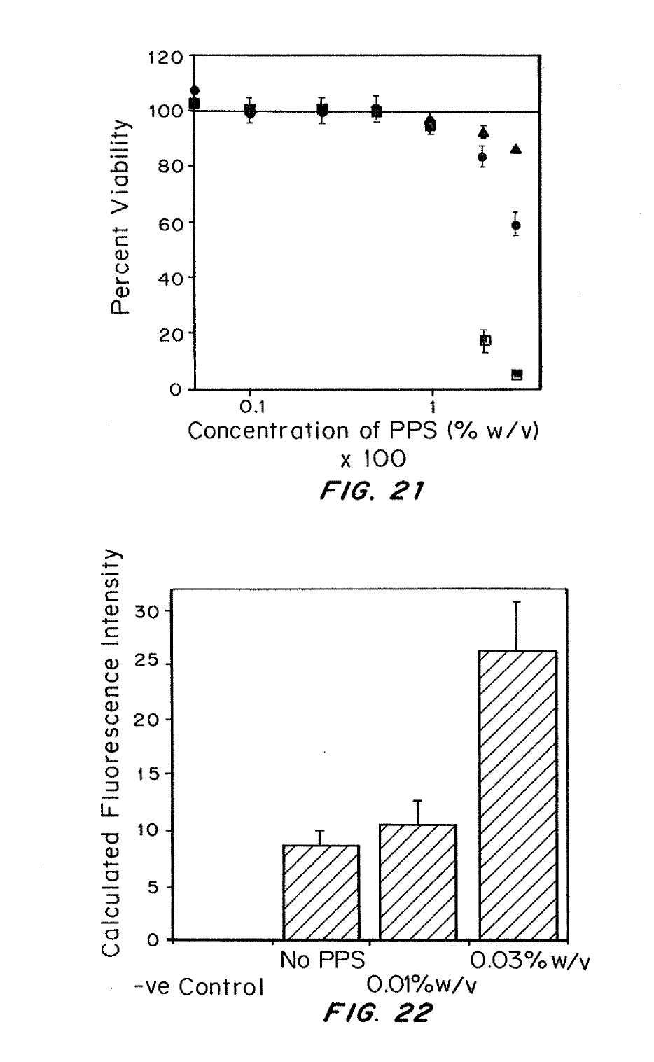

Toxicity assay showed that PPS is a very safe CPE with no detectable toxicity on Caco-2 cells up to 0.01% w/v, regardless of the exposure time, with exposure-dependent toxicity at higher concentrations. A 10 minute exposure to 0.03% w/v PPS solution resulted in a cell viability of ˜88% which further decreased with increase in exposure time (see FIG. 21). Cytotoxicity data suggest no mitochondrial toxicity of PPS despite TEER reduction, which may be due to changes in plasma membrane resistance.

Macromolecule (FITC-Insulin) Transport Quantification with Confocal Microscopy

Confocal microscopic imaging further confirmed the permeation enhancement effects of PPS. As noted in FIG. 22, PPS exposure significantly increased FITC-insulin transport across the monolayer (1.3 fold for 0.01% and 3.1 fold for 0.03% w/v PPS solution), which is in congruence with the transwell studies suggestive of enhanced permeability via both paracellular and transcellular route.

Applications of PPS in Intestinal Delivery of Salmon Calcitonin (sCT), a Therapeutic Peptide with Very Poor Oral Bioavailability

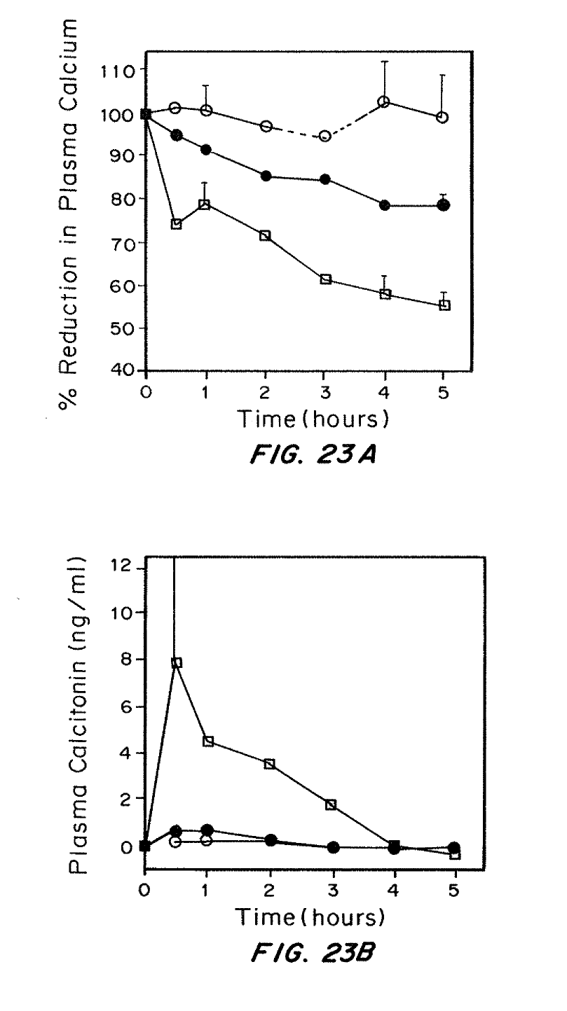

In vivo efficacy of PPS was tested in SD rats by analyzing pharmacokinetic and pharmacodynamic profiles of salmon calcitonin (sCT), a very poorly permeable therapeutic peptide. Two doses of PPS, 0.1 and 1% w/v were tested in vivo. These doses were higher than those used in vitro to account for the fact that PPS is delivered over a larger area in vivo and its effect is likely mitigated by the presence of serum and mucus proteins. Intestinal injection of 3 mg/kg sCT (negative control) solution in the absence of PPS did not produce significant reduction in plasma calcium, whereas incorporation of PPS provided concentration-dependent reduction in plasma calcium (˜80% for 0.1% PPS, and ˜56% for 1% PPS as compared to the initial values) (FIG. 23A). Pharmacokinetic profile matched pharmacodynamic observations, with intestinal administration of sCT (negative control, 3 mg/kg) delivered negligible amounts of sCT in blood, with PPS incorporation enhancing systemic absorption of sCT in a dose-dependent manner (FIG. 23B). In fact, 1% w/v PPS led to a ˜45-50-fold enhancement of systemic sCT availability based on peak plasma concentrations.

It was also confirmed that neither 0.1% nor 1% w/v PPS solution caused pathological damage to the intestinal epithelium. Intestinal structure exposed to both the PPS concentrations, 0.1% and 1% w/v, was comparable to negative control (sterile saline injection) in terms of microscopic appearance of intestinal epithelium with no significant presence of inflammatory cells or erosion, and no evidences of necrosis or specific inflammation. Epithelial layers were intact with any disruption, and the villus structure was relatively normal as well. Cellularity of the tissue was not significantly changed due to PPS injection.

These data suggest that PPS is a safe and potent enhancer of intestinal transport of therapeutic macromolecules, which provided significant enhancement of sCT transport at a fraction of dose of currently investigated CPEs.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A composition comprising a drug to be delivered and one or more chemical permeation enhancers, wherein the chemical permeation enhancers have an overall potential (OP) of at least 0.5.

2. The composition of claim 1, wherein the one or more chemical permeation enhancers are present in a concentration effective to increase the rate of absorption of the drug at a site of delivery, relative to rate of absorption of the drug at the same site in the absence of the chemical permeation enhancer, without causing necrosis or specific inflammation at the site of delivery.

3. The composition of claim 1, wherein the one or more chemical permeation enhancers are present in a concentration effective to increase the rate of absorption of the drug at a site of delivery, relative to rate of absorption of the drug at the same site in the absence of the chemical permeation enhancer, without causing one or more symptoms associated with malfunctions of the gastrointestinal tract.

4. The composition of claim 1, wherein the composition is in a form selected from the group consisting of gels, solutions, creams, sprays, powders and tablets.

5. The composition of claim 1, wherein the chemical permeation enhancer has a preferential ability to deliver drugs into epithelial cells.

6. The composition of claim 1, wherein the chemical permeation enhancer is a zwitterionic surfactant.

7. The composition of claim 6, wherein the chemical permeation enhancer is palmityldimethyl ammonio propane sulfonate (PPS) or a structural analog thereof.

8. The composition of claim 1, wherein the chemical permeation enhancer is a nonionic surfactant, such as polysorbate 20, 40, 60, or 80.

9. The composition of claim 2, wherein the site of delivery is in a mucosal layer is selected from the group consisting of mucosa of the intestine, colon, oral cavity and nasal cavity.

10. The composition of claim 1, wherein the drug is a protein or a peptide.

11. The composition of claim 7, wherein the drug is insulin or an analog thereof.

12. A method of enhancing mucosal drug delivery, comprising administering to a patient in need thereof a composition comprising a drug to be delivered and one or more chemical permeation enhancers, wherein the chemical permeation enhancers have an overall potential (OP) of at least 0.5.

13. The method of claim 12, wherein the one or more chemical permeation enhancers are present in a concentration effective to increase the rate of absorption of the drug at a site of delivery, relative to rate of absorption of the drug at the same site in the absence of the chemical permeation enhancer, without causing necrosis or specific inflammation at the site of delivery.

14. The method of claim 13, wherein the site of delivery is in a mucosal layer is selected from the group consisting of mucosa of the intestine, colon, oral cavity and nasal cavity.

15. The method of claim 12, wherein the chemical permeation enhancer has a preferential ability to deliver drugs into epithelial cells.

16. The method of claim 12, wherein the chemical permeation enhancer is a zwitterionic surfactant.

17. The method of claim 16, wherein the chemical permeation enhancer is palmityldimethyl ammonio propane sulfonate (PPS) or a structural analog thereof.

18. The method of claim 12, wherein the chemical permeation enhancer is a nonionic surfactant, such as polysorbate 20, 40, 60, or 80.
